(19) United States
(12) Patent Application Publication
(10) Pub. No.: US 2025/0333790 A1
(43) Pub. Date: Oct. 30, 2025

(54) SCREENING METHOD FOR RHEUMATOID ARTHRITIS

(71) Applicant: AGE LABS AS, Oslo (NO)

(72) Inventors: Espen RISKEDAL, Oslo (NO); Karl Trygve KALLEBERG, Oslo (NO); Arne SØRAAS, Oslo (NO); Cathrine Lund HADLEY, Oslo (NO); Janis Frederick NEUMANN, Oslo (NO)

(21) Appl. No.: 18/696,669

(22) PCT Filed: Oct. 4, 2022
(86) PCT No.: PCT/EP2022/077612

§ 371 Date: Mar. 28, 2024

(30) Foreign Application Priority Data
Oct. 4, 2021 (EP) 21200767.8

(51) Int. Cl. C12Q 1/6883; G16H 50/20

(52) U.S. Cl. CPC C12Q 1/6883; G16H 50/20; C12Q 2600/154

(57) ABSTRACT

The present invention relates generally to methods of screening for rheumatoid arthritis, as well as kits for screening for rheumatoid arthritis. More particularly, the invention relates to a method of screening for rheumatoid arthritis in a subject, the method comprising using methylation levels of CpG sites in DNA from a biological sample obtained from the subject in order to screen for rheumatoid arthritis in the subject, wherein said methylation levels are used to provide an indication of the presence or absence of rheumatoid arthritis in the subject.

The present invention relates generally to methods of screening for rheumatoid arthritis, as well as kits for screening for rheumatoid arthritis.

Rheumatoid arthritis (RA) is a long-term autoimmune disorder that primarily affects the joints. Diagnosing Rheumatoid Arthritis is clearly defined in the ACR/EULAR 2010 rheumatoid arthritis classification criteria and is followed by rheumatologists worldwide. There are four domains, with point scores for each: joint symptoms; serology (including rheumatoid factor (RF) and/or anti-citrullinated protein antibody (ACPA)); symptom duration, whether <6 weeks or >6 weeks; and acute-phase reactants (CRP and/or ESR). The points from each domain are added and the sum is considered to be the total score. A total score of >6 is needed to classify a patient as having definite RA. Essentially, four lab tests are used when diagnosing RA: screening for RF (Rheumatoid factor), an autoantibody associated with RA and other autoimmune diseases; screening for ACPA, an autoantibody present in the majority of RA patients; screening for CRP (C-reactive protein), a protein found in blood plasma in response to inflammation; and determination of ESR (Erythrocyte sedimentation rate), the rate at which red blood cells descend in a standardized tube over time, for a measure of inflammation.

The two most common lab tests used for diagnosing rheumatoid arthritis are the ACPA and RF screening tests. A drawback with both of these tests is their propensity to yield false positives on diseases similar to rheumatoid arthritis. This is because ACPA and RF are not biomarkers exclusively for rheumatoid arthritis. In between 20 to 80% of RF-positive cases, and up to 10% of ACPA-positive cases, the subject does not have rheumatoid arthritis but rather has a similar disease, usually an autoimmune or autoinflammatory disease. The subject could alternatively have an another arthritic disease.

One alternative type of diagnostic test that can be used to try and detect the presence or absence of RA is methylation screening. This involves detecting the methylation level of a number of CpG sites in DNA (e.g. genomic DNA) from a biological sample obtained from the subject, for example from a blood sample. From this combination of methylation levels, a diagnosis can be made of whether the subject has or does not have RA. The quality of the diagnostic test depends upon the selection of CpG sites that are analysed, since certain CpG sites will be more relevant indicators of disease status than others.

Rhead et al. 2017 (“Rheumatoid arthritis naive T cells share hypermethylation sites with synoviocytes.” Arthritis & Rheumatology 69.3: 550-559) trained an algorithm that from 79 CpG sites could classify between rheumatoid arthritis and controls with an AUC of 0.807. The CpG sites were found in a study using Fibroblast-like synoviocytes (FLS) from RA patients, but still worked when testing on CD4+ naive T cells from GSE131989 (63 RA and 31 healthy controls). Ambatipudi et al. 2018 (“Assessing the role of DNA methylation-derived neutrophil-to-lymphocyte ratio in rheumatoid arthritis.” Journal of immunology research) used an algorithm that from a methylation derived estimate of neutrophil-to-lymphocyte ratio (mdNLR) could classify between rheumatoid arthritis and controls with an AUC of 0.80 on dataset GSE42861.

The algorithms of the prior art mentioned above are trained on only one dataset using only a few hundred samples. In molecular biology, a batch effect occurs when non-biological factors in an experiment cause changes in the data produced by the experiment. Such effects can lead to inaccurate conclusions when their causes are correlated with one or more outcomes of interest in an experiment.

Additionally, those single datasets included only rheumatoid arthritis subjects or healthy subjects. Thus, the CpG sites identified in these tests were selected based only on the ability of those tests to distinguish between subjects having RA and healthy subjects.

Therefore, there remains a need for a method of screening for rheumatoid arthritis which reliably detects rheumatoid arthritis, while also being able to distinguish RA from diseases similar to RA, e.g. to have fewer false positives.

In the present invention the inventors have identified a selection of 145 CpG sites (Table 9) whose methylation levels are indicative of the presence or absence of rheumatoid arthritis. Unlike the methods of the prior art as discussed above, these CpG sites have been identified by training models using datasets containing methylation data from not only healthy and rheumatoid arthritis subjects, but also subjects which do not have RA but have diseases similar to RA.

Additionally, the RA test of the present invention has been trained on multiple datasets. This enables correction for batch effects, in contrast to the models of the prior art mentioned above, which were trained on only one dataset.

Thus, the method of screening of the invention is not only of high quality in distinguishing rheumatoid arthritis patients from healthy patients but also RA patients from patients having diseases similar to RA, for example other autoimmune and/or autoinflammatory diseases, and other arthritises. The present method of screening using CpG sites selected from this unique set of 145 CpG sites (Table 9) not only renders the screening method of the invention a surprising alternative method of screening for rheumatoid arthritis, but in fact a surprising improvement over the prior art as it provides a focussed RA detector as opposed to for example a generic inflammation detector that cannot distinguish well between RA and other diseases similar to RA.

Those CpG sites belonging to the list of 121 CpG sites of Table 3 are especially suitable to measure in order to discriminate between RA and other autoimmune and/or autoinflammatory diseases. A test using those 121 CpG sites provided herein surpasses either the sensitivity or the specificity of existing solutions depending on the selected threshold (see Table 4). The test provided herein is of high quality even when only smaller subsets of the 121 CpG sites are used. For example, it is demonstrated herein that AUC values of more than 0.9 can be achieved using 31 of the 121 CpG sites or even fewer, with an upper AUC value of greater than 0.95 when all 121 sites are used.

Similarly, tests using the 24 CpG sites provided in Tables 5, 6 and 7 achieve excellent results. For example, it is demonstrated herein that these 24 CpG sites are especially suitable to measure in order to discriminate between RA and other forms of arthritis, e.g. polyarthritis, reactive arthritis or psoriatic arthritis (PsA), as well as to discriminate between RA and healthy controls. The 24 CpG sites are also especially suitable for detecting seronegative RA—a subtype of RA which cannot be detected using the conventional screening methods of the art as mentioned above. 5 of the list of 24 CpG sites are also demonstrated herein to be especially useful in discriminating both RA-related inflammatory diseases and arthritises.

Thus, the CpG sites of the invention can in some embodiments advantageously be used to detect seronegative RA, and/or to discriminate between RA and other autoimmune and/or autoinflammatory diseases, and/or to discriminate between RA and healthy controls, and/or to discriminate between RA and other forms of arthritis, e.g. polyarthritis, reactive arthritis or psoriatic arthritis (PsA).

In one aspect, the invention provides a method of screening for rheumatoid arthritis in a subject (or a method of diagnosing rheumatoid arthritis in a subject, or a method of obtaining an indication of the presence or absence of rheumatoid arthritis in a subject, or a method of obtaining an indication of the course of rheumatoid arthritis in a subject, or a method of obtaining clinically relevant information about a subject), the method comprising using the methylation levels of at least;
1, 4, 5, 10, 15, 20, 23, 25, 30, 40, 50, 60, 70, 80, 90, 100, 110, 120, 121, 130, 140 or 145 CpG sites selected from the list of CpG site numbers 1 to 145 of Table 9; in DNA from a biological sample obtained from the subject in order to screen (etc.)

In another aspect, the invention provides a method of screening for rheumatoid arthritis in a subject, the method comprising using the methylation levels of at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from the subject in order to screen for rheumatoid arthritis in the subject, wherein said methylation levels are used to provide an indication of the presence or absence of rheumatoid arthritis in the subject.

In another aspect, the invention provides a method of diagnosing rheumatoid arthritis in a subject, the method comprising using the methylation levels of at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from the subject in order to diagnose rheumatoid arthritis in the subject, wherein said methylation levels are used to provide an indication of the presence or absence of rheumatoid arthritis in the subject.

In another aspect, the invention provides a method of obtaining an indication of the presence or absence of rheumatoid arthritis in a subject, the method comprising using the methylation levels of at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from the subject in order to screen for rheumatoid arthritis in the subject, wherein said methylation levels are used to provide an indication of the presence or absence of rheumatoid arthritis in the subject.

In another aspect, the invention provides a method of obtaining an indication of the course of rheumatoid arthritis in a subject, the method comprising using the methylation levels of at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from the subject in order to obtain an indication of the course of rheumatoid arthritis in the subject, wherein said methylation levels are used to provide an indication of the presence or absence of rheumatoid arthritis in the subject.

In another aspect, the invention provides a method of obtaining clinically relevant information about a subject (preferably a subject suspected of having rheumatoid arthritis), the method comprising using the methylation levels of at least or at most:
1, 4, 5, 10, 11, 15, 20, 25, 30, 31, 40, 50, 60, 70, 80, 90, 100, 110, 120, or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from the subject in order to obtain clinically relevant information about the subject, wherein said methylation levels are used as an indication of clinically relevant information about the subject (or as an indication of the presence or absence of rheumatoid arthritis in the subject).

In any aspects and embodiments of the methods (or kits or computer programs, etc.) herein, the using of CpG site(s) selected from the 121 CpG sites listed in Table 3 may preferably comprise using the methylation levels of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 CpG sites selected from the list CpG site numbers 1 to 121 of Table 3.

In any aspects or embodiments of the methods (or kits or computer programs, etc.) herein, the using of CpG site(s) selected from the 121 CpG sites listed in Table 3 may preferably comprise using the methylation levels of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 CpG sites selected from the list of CpG site numbers 1 to 31 in Table 3. In preferred embodiments, the method (or kit or computer program, etc.) uses at least or at most 1, 5, 10, 15, 20, 25, 30, or 31 CpG sites selected from the list of CpG site numbers 1 to 31 in Table 3.

In any aspects or embodiments of the methods (or kits or computer programs, etc.) herein, the using of CpG site(s) selected from the 121 CpG sites listed in Table 3 may preferably comprise using the methylation levels of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG sites selected from the list of CpG site numbers 32 to 61 in Table 3. In preferred embodiments, the method (or kit or computer program, etc.) uses at least or at most 1, 5, 10, 15, 20, 25, or 30 CpG sites selected from the list of CpG site numbers 32 to 61 in Table 3.

In any aspects or embodiments of the methods (or kits or computer programs, etc.) herein, the using of CpG site(s) selected from the 121 CpG sites listed in Table 3 may preferably comprise using the methylation levels of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG sites selected from the list of CpG site numbers 62 to 91 in Table 3. In preferred embodiments, the method (or kit or computer program, etc.) uses at least or at most 1, 5, 10, 15, 20, 25, or 30 CpG sites selected from the list of CpG site numbers 62 to 91 in Table 3.

In any aspects or embodiments of the methods herein, the using of CpG site(s) selected from the 121 CpG sites listed in Table 3 may preferably comprise using the methylation levels of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG sites selected from the list of CpG site numbers 92 to 121 in Table 3. In preferred embodiments, the method (or kit or computer program, etc.) uses at least or at most 5, 10, 15, 20, 25, or 30 CpG sites selected from the list of CpG site numbers 92 to 121 in Table 3.

In any aspects or embodiments of the methods (or kits or computer programs, etc.) herein, the using of CpG site(s) selected from the 121 CpG sites listed in Table 3 may preferably comprise using the methylation levels of at least or at most the CpG sites referred to in Table 3 as CpG site numbers 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2 (i.e. at least or at most CpG site numbers 1 and 2 of Table 3), or 1 (i.e. at least or at most CpG site number 1 of Table 3).

In any aspects or embodiments of the methods (or kits or computer programs, etc.) herein, the using of CpG site(s) selected from the 24 CpG sites listed in Table 5 may preferably comprise using the methylation levels of at least or at most the CpG sites referred to in Table 5 as CpG site numbers 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 1, 1 to 10, 1 to 9, 1 to 8, 1 to 7, 1 to 6, 1 to 5, 1 to 4, 1 to 3, 1 to 2 (i.e. at least or at most CpG site numbers 1 and 2 of Table 5), or 1 (i.e. at least or at most CpG site number 1 of Table 5). More preferably, the using of CpG site(s) selected from the 24 CpG sites listed in Table 5 comprises using the methylation levels of at least or at most the CpG sites referred to in Table 5 as CpG site numbers 1 to 18.

In any aspects or embodiments of the methods (or kits or computer programs, etc.) herein, the using of CpG site(s) selected from the 20 CpG sites listed in Table 6 may preferably comprise using the methylation levels of at least or at most the CpG sites referred to in Table 5 as CpG site numbers 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2 (i.e. at least or at most CpG site numbers 1 and 2 of Table 6), or 1 (i.e. at least or at most CpG site number 1 of Table 6). More preferably, the using of CpG site(s) selected from the 20 CpG sites listed in Table 6 comprises using the methylation levels of at least or at most the CpG sites referred to in Table 5 as CpG site numbers 1 to 9, 1 to 5, or 1 to 3.

In any aspects or embodiments of the methods (or kits or computer programs, etc.) herein, the using of CpG site(s) selected from the 16 CpG sites listed in Table 7 may preferably comprise using the methylation levels of at least or at most the CpG sites referred to in Table 7 as CpG site numbers 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2 (i.e. at least or at most CpG site numbers 1 and 2 of Table 7), or 1 (i.e. at least or at most CpG site number 1 of Table 7). More preferably, the using of CpG site(s) selected from the 16 CpG sites listed in Table 7 comprises using the methylation levels of at least or at most the CpG sites referred to in Table 7 as CpG site numbers 1 to 12, 1 to 9, or 1 to 6.

In any aspects or embodiments of the methods (or kits or computer programs, etc.) herein, in some aspects or embodiments one, two, three, four or all five of the following CpG sites are not used (or measured or targeted): cg04399899, cg07329251, cg07930752, cg10266904, and cg27552857. Alternatively viewed, in some aspects or embodiments cg04399899 is not used (or measured or targeted); or cg07329251 is not used (or measured or targeted); or cg07930752 is not used (or measured or targeted); or cg10266904 is not used (or measured or targeted); and/or cg27552857 is not used (or measured or targeted).

In any aspects or embodiments of the methods herein, the using of CpG site(s) selected from the 24 CpG sites listed in Table 5 may preferably comprise using the methylation levels of at least or at most:
1, 2, 3, 4, 5, 6, 7, 8, 9, 10, 11, 12, 13, 14, 15, 16, 17, 18, 19 or 20 CpG sites selected from the list of CpG site numbers 1 to 20 of Table 6; and/or
1, 2, 3, 4, 5, 6, 7, 8, 9, 10, 11, 12, 13, 14, 15 or 16 CpG sites selected from the list of CpG site numbers 1 to 16 of Table 7.

In any aspects or embodiments of the methods (or kits or computer programs, etc.) herein, the method (or kit or computer program, etc.) preferably uses the methylation levels of at least:
10 CpG sites selected from the list of CpG site numbers 1 to 145 of Table 9.

In any aspects or embodiments of the methods (or kits or computer programs, etc.) herein, the method (or kit or computer program, etc.) more preferably uses the methylation levels of at least:
4 CpG sites selected from the list of CpG site numbers 1 to 121 of Table 3; and/or
10 CpG sites selected from the list of CpG site numbers 1 to 24 of Table 5.

In any aspects or embodiments of the methods (or kits or computer programs, etc.) herein, the method (or kit or computer program, etc.) more preferably uses the methylation levels of at least:
11 CpG sites selected from the list of CpG site numbers 1 to 121 of Table 3.

Alternatively viewed, the invention provides a method of screening for rheumatoid arthritis in a subject (or a method of diagnosing rheumatoid arthritis in a subject, or a method of obtaining an indication of the presence or absence of rheumatoid arthritis in a subject, or a method of obtaining an indication of the course of rheumatoid arthritis in a subject, or a method of obtaining clinically relevant information about a subject), the method comprising using the methylation levels of at least or at most:
1, 4, 5, 10, 11, 15, 20, 25, 30, 31, 40, 50, 60, 70, 80, 90, 100, 110, 120 or 121 CpG sites selected from the list of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from the subject in order to screen, etc., for rheumatoid arthritis in the subject, wherein said methylation levels are indicative of, or used as an indication of, or used to provide an indication of, the presence or absence of rheumatoid arthritis, etc., in the subject.

In another aspect, the invention provides a method of screening for rheumatoid arthritis in a subject (or a method of diagnosing rheumatoid arthritis in a subject, or a method of obtaining an indication of the presence or absence of rheumatoid arthritis in a subject, or a method of obtaining an indication of the course of rheumatoid arthritis in a subject, or a method of obtaining clinically relevant information about a subject), the method comprising using the methylation levels of a set of CpG sites in DNA from a biological sample obtained from the subject in order to screen, etc., for rheumatoid arthritis in the subject, wherein said methylation levels are indicative of the presence or absence of rheumatoid arthritis in the subject, and wherein said set of CpG sites comprises CpG sites in (or from or located in) at least or at most 1, 5, 10, 15, 20, 25, 30, 35, 40, 45, 50, 55, 60, 65, 70, 75, 80, 85, 90, 95 or 98 of the genes listed in the column “UCSC_RefGene_Name” in Table 3.

In embodiments, the set of CpG sites comprises CpG sites in (or from or located in) at least or at most 1, 2, 3, 4, 5, 6, 7, 8, 9, 10, 11, 12, 13, 14, 15, 16, 17, 18, 19, 20 or 21 of the following genes: NLRC5, SMARCA4, HLA-DQA2, SAFB, SAFB2, SMU1, BCAS4, TH, KIF16B, PVT1, NCALD, CD28, ALDH16A1, CNNM2, HOXB9, E4F1, MICAL1, LOC285768, INSM1, SNORD116-24 and GALNT2. Optionally, the set of CpG sites comprises (or further comprises) any of the CpG sites or combinations of CpG sites of Table 3, for example as contemplated above.

In embodiments, the set of CpG sites comprises CpG sites in (or from or located in) at least or at most 1, 2, 3, 4, 5, 6, 7, 8, 9, 10, 11, 12, 13, 14 or 15 of the following genes: NLRC5, SMARCA4, HLA-DQA2, SAFB, SAFB2, SMU1, BCAS4, TH, KIF16B, PVT1, NCALD, CD28, ALDH16A1, CNNM2 and HOXB9. Optionally, the set of CpG sites comprises (or further comprises) any of the CpG sites or combinations of CpG sites of Table 3, for example as contemplated above.

In embodiments, the set of CpG sites comprises CpG sites in (or from or located in) at least or at most 1, 2, 3, 4, 5, 6, 7, 8 or 9 of the following genes: NLRC5, SMARCA4, HLA-DQA2, SAFB, SAFB2, SMU1, BCAS4, TH and KIF16B. Optionally, the set of CpG sites comprises (or further comprises) any of the CpG sites or combinations of CpG sites of Table 3, for example as contemplated above.

In embodiments, the set of CpG sites comprises CpG sites in (or from or located in) at least or at most 1, 2, 3, 4, 5 or 6 of the following genes: NLRC5, SMARCA4, HLA-DQA2, SAFB, SAFB2, SMU1. Optionally, the set of CpG sites comprises (or further comprises) any of the CpG sites or combinations of CpG sites of Table 3, for example as contemplated above.

In embodiments, the set of CpG sites comprises CpG sites in (or from or located in) at least or at most 1, 2, 3, 4, 5 or 6 of the following genes: HLA-DQA1, ELANE, HLA-DQA2, HLA-DQB1, CD28 and CD1C. Optionally, the set of CpG sites comprises (or further comprises) any of the CpG sites or combinations of CpG sites of Table 3, for example as contemplated above.

In embodiments, the set of CpG sites comprises CpG sites in (or from or located in) at least or at most 1, 2, 3, 4, 5, 6, 7, 8, 9, 10, 11, 12, 13, 14, 15, 16, 17, 18, 19, 20, 21, 22, 23, 24, 25 or 26 of the following genes NLRC5, SMARCA4, HLA-DQA2, SAFB, SAFB2, SMU1, BCAS4, TH, KIF16B, PVT1, NCALD, CD28, ALDH16A1, CNNM2, HOXB9, E4F1, MICAL1, LOC285768, INSM1, SNORD116-24, GALNT2, HLA-DQA1, ELANE, HLA-DQB1, CD28 and CD1C. Optionally, the set of CpG sites comprises (or further comprises) any of the CpG sites or combinations of CpG sites of Table 3, for example as contemplated above.

In alternative embodiments, the CpG sites of the sets of CpG sites can be “associated with” any of the genes or lists of genes recited above. In alternative embodiments, the CpG sites of the sets of CpG sites can be “associated with” and/or “in” any of the genes or lists of genes recited above.

The genes provided in Table 3 are genes found in humans.

As used herein, where reference is made to “using” or “measuring” methylation levels, the acts of “observing”, “obtaining”, “determining”, “detecting” and/or “assessing” said methylation levels are contemplated alternatively or in addition. All the terms quoted in this paragraph may be used interchangeably if appropriate.

As used herein, where it is recited that methylation levels are “indicative of the presence or absence of rheumatoid arthritis in the subject” or “used to provide an indication of the presence or absence of rheumatoid arthritis in the subject” or other similar terms, it is meant that there is a positive correlation between the methylation levels and the presence of rheumatoid arthritis in that subject.

Where the term “measuring” in respect of methylation levels is recited, the term “selectively measuring” is also encompassed thereby.

The phrase “selectively measuring” as used herein refers to methods wherein the methylation levels of only a finite number of CpG sites are measured rather than measuring the methylation levels essentially of all or essentially all potential CpG sites in a genome. For example, in some aspects, “selectively measuring” methylation levels can refer to measuring the methylation levels of no more than 100000, 90000, 80000, 70000, 60000, 50000, 40000, 30000, 20000, 10000, 9000, 8000, 7000, 6000, 5000, 4000, 3000, 2000, 1000, 900, 800, 700, 600, 500, 400, 300, 200, 3, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 or 121 different CpG sites.

Similarly, where the term “using” in respect of methylation levels is recited, the term “selectively using” is also encompassed thereby.

The phrase “selectively using” as used herein refers to methods wherein the methylation levels of only a finite number of CpG sites are used rather than using the methylation levels of all or essentially all potential CpG sites in a genome. For example, in some aspects, “selectively using” methylation levels can refer to using the methylation levels of no more than 100000, 90000, 80000, 70000, 60000, 50000, 40000, 30000, 20000, 10000, 9000, 8000, 7000, 6000, 5000, 4000, 3000, 2000, 1000, 900, 800, 700, 600, 500, 400, 300, 200, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 different CpG sites.

Where the term “detecting” in respect of methylation levels is recited, the term “selectively detecting” is also encompassed thereby.

The phrase “selectively detecting” as used herein refers to methods wherein the methylation levels of only a finite number of CpG sites are measured rather than measuring the methylation levels essentially of all or essentially all potential CpG sites in a genome. For example, in some aspects, “selectively detecting” methylation levels can refer to detecting the methylation levels of no more than 100000, 90000, 80000, 70000, 60000, 50000, 40000, 30000, 20000, 10000, 9000, 8000, 7000, 6000, 5000, 4000, 3000, 2000, 1000, 900, 800, 700, 600, 500, 400, 300, 200, 3, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 different CpG sites.

As discussed herein, methods of the present invention may comprise using, determining or measuring, etc., the methylation levels of one or more CpG sites “selected from the list of” certain specific CpG sites set forth herein, or may comprise using, determining or measuring, etc., the methylation levels of one or more CpG sites belonging to one or more genes “selected from the list of” certain specific genes set forth herein. For the avoidance of doubt, in some embodiments in which the methylation levels—of one or more of the specific CpG sites “selected from the list” set forth herein, or of one or more CpG sites belonging to one or more genes “selected from the list of” certain specific genes set forth herein—are used, measured or determined, etc., the methylation levels of one or more other (or distinct or alternative) CpG sites, or of one or more other (or distinct or alternative) CpG sites belonging to one or more other genes, and/or one or more other biomarkers, may additionally be used, measured or determined. Thus, “selected from the list of” may be an “open” term. In other embodiments, the methylation levels of only one or more of the specific CpG sites discussed herein is used, measured or determined, etc. (e.g. the methylation levels of other CpG sites or other biomarkers are not used, measured or determined).

As used herein, the term “CpG site” is given its art recognised meaning and refers to the location in a nucleic acid molecule, or sequence representation of the molecule, where a cytosine nucleotide and guanine nucleotide occur, the 3′ oxygen of the cytosine nucleotide being covalently attached to the 5′ phosphate of the guanine nucleotide. The nucleic acid is typically DNA. The cytosine nucleotide can optionally be methylated at position 5 of the pyrimidine ring. Such CpG sites can be referred to as methylated CpG sites. Unless otherwise stated, nucleic acid sequences recited herein are recited in the 5′ to 3′ direction.

As used herein, the term “methylation level” includes the average methylation state of a CpG site in a biological sample. Methylation levels of each CpG site may be quantified by methods known in the art, for example in the form of a beta value or M value. When measuring DNA methylation using microarray technology (such as HumanMethylation450 BeadChip array, which covers approximately 450,000 CpG sites), the beta value is the ratio of the methylated probe intensity and the overall intensity (sum of methylated and unmethylated probe intensities). The beta-value is thus generally and conveniently a number between 0 and 1, or 0 and 100%. A value of zero indicates that all copies of the CpG site in the sample were completely unmethylated (no methylated molecules were measured) and a value of one (or 100%) indicates that every copy of the CpG site in the sample was methylated.

In embodiments, the methylation levels referred to herein are methylation states. The “methylation state” of a particular CpG site in a particular cell is either methylated or non-methylated.

In general, the methods of the invention are carried out in vitro or ex vivo (unless the context requires otherwise, e.g. administration steps).

Throughout the aspects and embodiments provided herein, it will be appreciated that the methylation levels of any number of the 145 CpG sites listed in Table 9 could be used, i.e. the methylation levels of at least or at most or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 of the CpG sites recited in Table 9.

Throughout the aspects and embodiments provided herein, it will be appreciated that any particular selection of the 145 CpG sites listed in Table 9 could be used, i.e. the methylation level of CpG site number 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 and/or 145 as named in Table 9.

Throughout the aspects and embodiments provided herein, it will be appreciated that the methylation levels of any number of the 121 CpG sites listed in Table 3 could be used, i.e. the methylation levels of at least or at most or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 or 121 of the CpG sites recited in Table 3.

Throughout the aspects and embodiments provided herein, it will be appreciated that any particular selection of the 121 CpG sites listed in Table 3 could be used, i.e. the methylation level of CpG site number 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 and/or 121 as named in Table 3.

Throughout the aspects and embodiments provided herein, it will be appreciated that the methylation levels of any number of the 24 CpG sites listed in Table 5 could be used, i.e. the methylation levels of at least or at most or exactly 1, 2, 3, 4, 5, 6, 7, 8, 9, 10, 11, 12, 13, 14, 15, 16, 17, 18, 19, 20, 21, 22, 23 or 24 of the CpG sites recited in Table 5.

Throughout the aspects and embodiments provided herein, it will be appreciated that any particular selection of the 24 CpG sites listed in Table 5 could be used, i.e. the methylation level of CpG site number 1, 2, 3, 4, 5, 6, 7, 8, 9, 10, 11, 12, 13, 14, 15, 16, 17, 18, 19, 20, 21, 22, 23 and/or 24 as named in Table 5.

Throughout the aspects and embodiments provided herein, it will be appreciated that the methylation levels of any number of the 20 CpG sites listed in Table 6 could be used, i.e. the methylation levels of at least or at most or exactly 1, 2, 3, 4, 5, 6, 7, 8, 9, 10, 11, 12, 13, 14, 15, 16, 17, 18, 19 or 20 of the CpG sites recited in Table 6.

Throughout the aspects and embodiments provided herein, it will be appreciated that any particular selection of the 20 CpG sites listed in Table 6 could be used, i.e. the methylation level of CpG site number 1, 2, 3, 4, 5, 6, 7, 8, 9, 10, 11, 12, 13, 14, 15, 16, 17, 18, 19 and/or 20 as named in Table 6.

Throughout the aspects and embodiments provided herein, it will be appreciated that the methylation levels of any number of the 16 CpG sites listed in Table 7 could be used, i.e. the methylation levels of at least or at most or exactly 1, 2, 3, 4, 5, 6, 7, 8, 9, 10, 11, 12, 13, 14, 15 or 16 of the CpG sites recited in Table 7.

Throughout the aspects and embodiments provided herein, it will be appreciated that any particular selection of the 16 CpG sites listed in Table 7 could be used, i.e. the methylation level of CpG site number 1, 2, 3, 4, 5, 6, 7, 8, 9, 10, 11, 12, 13, 14, 15 and/or 16 as named in Table 7.

Rheumatoid arthritis may be identified by the method of the present invention by relying only on measurements of methylation levels from the subject. However, it will also be appreciated that further variables may also be used and/or measured. Two such exemplary further variables are the presence/absence of rheumatoid factor (RF) in the subject (e.g. in the blood or a blood sample of the subject) and the presence/absence of anti-citrullinated protein antibodies (ACPA) (also known as anti-cyclic citrullinated peptide (anti-CCP) antibodies) in the subject (e.g. in the blood or a blood sample of the subject). These variables may be collectively referred to herein as serology data or serology information. In the method of the present invention, the presence of one or both of these two components may be used to support a diagnosis of the presence of rheumatoid arthritis in the subject, or support a diagnosis that the subject has rheumatoid arthritis (and conversely, the absence of one or both of these two components may be used to support the absence of rheumatoid arthritis in the subject, or support a diagnosis that the subject does not have rheumatoid arthritis).

Thus, in any of the aspects or embodiments of the methods herein, the method may comprise (or further comprise) using (or additionally using) the RF status of the subject (i.e. whether RF is present or absent in the subject, e.g. in the blood of the subject) and/or ACPA status of the subject (i.e. whether ACPA is present or absent in the subject, e.g. in the blood of the subject). In other words, the method may comprise using said RF status and/or ACPA status (in addition to said methylation levels) in order to provide the indication of the presence or absence of rheumatoid arthritis in the subject.

In particular, the inventors have identified a selection of CpG sites which are especially suitable for use in conjunction with serology data, e.g. RF status and/or ACPA status. These are the 16 CpG sites provided in Table 7.

Thus, in embodiments of the methods herein where CpG site(s) from the list of 16 CpG sites of Table 7 are used, the method may further comprise using the RF status of the subject (i.e. whether RF is present or absent in the subject, e.g. in the blood of the subject) and/or ACPA status of the subject (i.e. whether ACPA is present or absent in the subject, e.g. in the blood of the subject). Such embodiments may therefore optionally involve determining the RF and/or ACPA status of the subject. Alternatively, the RF and/or ACPA status may be already known or determined elsewhere.

Where reference is made to the use of methylation levels of CpG sites, it can also or alternatively be phrased or worded that the CpG sites themselves are used.

A diagnosis or diagnosing step (e.g. a step of diagnosing rheumatoid arthritis or the presence or absence or rheumatoid arthritis in a subject) can alternatively be worded as a classification or classification step (e.g. a step of classifying a subject as having or not having rheumatoid arthritis). The classification or diagnosing can be achieved by assignment of a cutoff value as described elsewhere herein.

The terms “likelihood” and “probability” and “p” can be used interchangeably herein.

In embodiments of any of the methods of the invention provided herein, the indication of the presence or absence of rheumatoid arthritis in the subject can be provided using machine learning (or a machine learning technique). In embodiments, the indication can be provided for example using appropriate techniques such as random forest, gradient boosting, a neural network, or linear or logistic regression.

Various scoring methods, scoring systems, markers or formulas can be used that comprise any appropriate combination of the CpG sites or methylation levels of the invention as described herein in order to arrive at an indication, e.g. in the form of a value or score, which can then be used for diagnosis of rheumatoid arthritis. For example, said methods etc., can be an algorithm that comprises any appropriate combination of the CpG sites or methylation levels as an input, to e.g. perform pattern recognition of the samples, in order to arrive at an indication, e.g. in the form of a value or score, which can then be used for diagnosis of rheumatoid arthritis. Non-limiting examples of such algorithms include machine learning algorithms that implement classification (algorithmic classifiers), such as linear classifiers (e.g. Fisher's linear discriminant, logistic regression, naive Bayes classifier, perceptron); support vector machines (e.g. least squares support vector machines); quadratic classifiers; kernel estimation (e.g. k-nearest neighbor); boosting (e.g. gradient boosting); decision trees (e.g. random forests); neural networks; and learning vector quantization.

The use of such classifiers, e.g. machine learning, random forest, gradient boosting or logistic regression, would be within the skill of a person skilled in the art. For example, such classifiers can conveniently be trained on methylation levels from a training set of samples and then tested in terms of accuracy on a test set of samples. The classifier may generate a black-box model that is trained on the most important methylation CpG sites or methylation levels.

In embodiments of any of the methods of the invention provided herein, the method comprises calculating a likelihood (or probability) of the subject having rheumatoid arthritis, for example as a function of said methylation levels.

The likelihood (or probability) can alternatively be referred to as likelihood value (or probability value). The likelihood (or probability) can be a value between 0 and 1. A value of 1 can indicate a 100% likelihood (or probability) that the subject has rheumatoid arthritis, and a value of 0 can indicate a 0% likelihood (or probability that the subject has rheumatoid arthritis). In preferred embodiments, the methods of the invention comprise calculating the likelihood as a function of a linear combination of said methylation levels, optionally to provide a value for (or representative of or corresponding to) the likelihood of the subject having rheumatoid arthritis.

In embodiments, the linear combination of said methylation levels comprises a weighted sum of said methylation levels, optionally to provide a value for (or representative of or corresponding to) the likelihood of the subject having rheumatoid arthritis.

Alternatively viewed, the weighted sum of methylation levels can be formed by applying a pre-determined weight (or coefficient) to each methylation value to provide a set of weighted methylation levels and then summing the weighted methylation levels, optionally to provide a value for (or representative of or corresponding to) the likelihood of the subject having rheumatoid arthritis.

In embodiments, a weight (or coefficient) as described herein is a normalised weight (or normalised coefficient), standardised weight (or standardised coefficient), or standardised logistic regression weight (or standardised logistic regression coefficient).

In embodiments, the method of the invention comprises calculating the likelihood as a logistic function of a linear combination of said methylation levels, optionally to provide a value for (or representative of or corresponding to) the likelihood of the subject having rheumatoid arthritis.

In embodiments, the method of the invention comprises performing a logistic regression method using said methylation levels, e.g. a linear combination of said methylation levels, optionally to provide a value for (or representative of or corresponding to) the likelihood of the subject having rheumatoid arthritis.

In embodiments, the method of the invention comprises receiving data representative of said methylation levels, and inputting the data to an algorithm for evaluating said function to determine the likelihood of the subject having rheumatoid arthritis.

In embodiments, the method comprises applying an algorithm (for example a statistical prediction algorithm) to the methylation levels, optionally in order to determine the rheumatoid arthritis disease status of the subject (or optionally to provide a value for (or representative of or corresponding to) the likelihood of the subject having rheumatoid arthritis).

In preferred embodiments, applying the algorithm, e.g. the statistical prediction algorithm, can comprise:
applying a weight (or coefficient), e.g. a pre-determined weight (or coefficient), to each methylation value to provide a set of weighted methylation levels;
summing the weighted methylation levels to provide a linear combination of methylation levels in the form of a weighted sum of said methylation levels; and
applying a logistic function to the weighted sum, optionally to provide a value for (or representative of or corresponding to) the likelihood of the subject having rheumatoid arthritis; and optionally
comparing the likelihood value (or likelihood) with a cutoff value (or cutoff).

In preferred embodiments of any of the methods provided herein, the weight (or coefficient), e.g. the pre-determined weight (or coefficient), for each methylation value has been calculated using reference methylation levels for each CpG site, wherein the reference methylation levels have been measured (or determined or obtained) from rheumatoid arthritis subjects (or observations of rheumatoid arthritis subjects) and from subjects not having rheumatoid arthritis (or observations of subjects not having rheumatoid arthritis). In preferred embodiments, the subjects not having rheumatoid arthritis comprise subjects having a disease similar to rheumatoid arthritis and optionally healthy subjects. In other embodiments, the subjects not having rheumatoid arthritis comprise or consist of healthy subjects.

Herein, a disease similar to rheumatoid arthritis can be any autoimmune and/or autoinflammatory disease that is not rheumatoid arthritis, and/or any arthritis (or arthritic disease) that is not rheumatoid arthritis. Preferably said autoimmune and/or autoinflammatory disease is one or more selected from the group consisting of coeliac disease, inflammatory bowel disease, systemic lupus erythematosus, aplastic anemia, myocarditis, lupus nephritis, autoimmune hepatitis, antisynthetase syndrome, psoriasis, scleroderma, vitiligo, Addison's disease, autoimmune polyendocrine syndrome (APS), autoimmune pancreatitis, diabetes mellitus type 1, autoimmune thyroiditis, Graves' disease, endometriosis, Sjogren syndrome, thrombocytopenia, Lyme disease, juvenile arthritis, palindromic rheumatism, psoriatic arthritis, fibromyalgia, myositis, myasthetina gravis, Guillan-Barre syndrome, autoimmune retinopathy, Meniere's disease, Behcet's disease, primary immunodeficiency, Crohn's disease, Ulcerative Colitis, Multiple Sclerosis, Pulmonary Tuberculosis, Sepsis, and Healthy Symptomatic Inflammatory Bowel Disease (IBD). More preferably said autoimmune and/or autoinflammatory disease is one or more selected from the group consisting of Crohn's disease, Ulcerative Colitis, Multiple Sclerosis, Pulmonary Tuberculosis, Sepsis, and Healthy Symptomatic Inflammatory Bowel Disease (IBD), and in some embodiments 2 or more, 3 or more, 4 or more, or 5 or more, or all, of these diseases. Preferably said arthritis is one or more selected from the group consisting of polyarthritis, reactive arthritis, psoriatic arthritis (PsA), osteoarthritis (OA), fibromyalgia and gout. More preferably said arthritis is one or more selected from the group consisting of polyarthritis, reactive arthritis and psoriatic arthritis (PsA), and in some embodiments 2 or more, or all, of these diseases.

In embodiments, the method comprises (or further comprises) comparing the likelihood or likelihood value with a cutoff or cutoff value (e.g. a pre-determined cutoff value). In embodiments, the method comprises (or further comprises) comparing the likelihood value with a cutoff value (e.g. a pre-determined cutoff value), wherein the likelihood value being above the cutoff value is indicative of the presence of rheumatoid arthritis in the subject and wherein the likelihood value being below the cutoff value is indicative of the absence of rheumatoid arthritis in the subject.

The comparing step may be considered to result in a diagnosis, i.e. of the presence or absence of rheumatoid arthritis in the subject. Alternatively viewed, the comparing step may be considered to result in a classification of the subject as having or not having rheumatoid arthritis.

In further embodiments, the method comprises (or further comprises) providing a readout or result indicating the presence or absence of rheumatoid arthritis based on the comparison of the likelihood (or likelihood value) with the cutoff (or cutoff value). In other words, the readout or result can be used as a diagnosis of the presence or absence of rheumatoid arthritis in the subject.

In the methods of the invention, appropriate threshold or cut-off scores or values can be calculated by methods known in the art, for example from the ROC curve, for use in the methods of the invention. Such cut-off scores or values or thresholds may be used to declare a sample positive or negative. Appropriate or optimal cut-off scores or values or thresholds can be calculated depending on the desired outcome of the method, for example a cut-off score or value or threshold can be determined (or selected) to maximise the accuracy of the assay. Alternatively or in addition, a cut-off score or value or threshold can be determined (or selected) to maximise the specificity of the assay, or the sensitivity of the assay, or both the sensitivity and the specificity of the assay (e.g. the maximum total sum of the sensitivity and specificity, or maximising the accuracy). Alternatively, a default cut-off can be used without calculation, for example a cut-off of 0.5 (in other words, a likelihood value of greater than 0.5 indicates rheumatoid arthritis). Appropriate cutoff values can readily be determined by a person skilled in the art as described elsewhere herein. However, exemplary cutoff values might be 0.5, 0.6, 0.7, 0.8, or 0.9.

Once the cut-off value has been determined, a sample whose likelihood score is below this threshold (cut-off) value is classified as not having rheumatoid arthritis, or, put another way, a sample whose likelihood score is above this cut-off value is classified as having rheumatoid arthritis. This way of determining threshold (cut-off) values could be used for any of the models (or algorithms) using different combinations of CpG sites described herein. Pre-determined or default cut-off values can also be used. Such threshold (cut-off) scores can then conveniently be used to assess the appropriate methylation data in subjects and to arrive at a diagnosis. Using an appropriate cut-off or threshold value (used to declare a sample positive or negative), models of the invention provided herein show outstanding results (AUC value of above 0.9). Thus, these results show that the present invention provides a simple and accessible test to allow accurate diagnosis of the presence of rheumatoid arthritis in an individual.

Good indicators of the performance of a diagnostic test are AUC, sensitivity, specificity, accuracy and balanced accuracy, especially AUC and balanced accuracy.

As used herein, the term “sensitivity” refers to the ability of the test to correctly identify those patients with the disease or disorder, such that a 100% sensitivity indicates a test that correctly identifies all patients with the disease or disorder. Sensitivity is calculated as: Sensitivity=(True Positives)/(True Positives+False Negatives). Sensitivity thus also provides a representation of the number of true positives or false negatives.

As used herein, the term “specificity” refers to the ability of a test to correctly identify those patients without the disease or disorder, such that a 100% specificity indicates a test that correctly identifies all patients without the disease or disorder. Specificity is calculated as: Specificity=(True Negatives)/(True Negatives+False Positives). Sensitivity thus also provides a representation of the number of true negatives or false positives.

As used herein, the term “accuracy” refers to the ability of a test to correctly identify the disease status (either having the disorder or not having the disorder) of each patient, such that 100% accuracy indicates a test that correctly identifies the disease status of every patient. Accuracy=correctly classified subjects/all subjects.

The “area under the receiver operating characteristic (ROC) curve” (AUC) is a global measure of diagnostic accuracy. The ROC curve is a plot of the pairs of sensitivity and specificity values for each cut-off, with 1-specificity (1 minus specificity) on the x-axis and sensitivity on the y-axis. Thus, while the sensitivity and specificity of a diagnostic test depend on the cut-off, the AUC is independent of cut-off. In some instances, AUC can therefore be more informative of the quality of a diagnostic test than sensitivity or specificity.

In general, an AUC of 0.5 suggests no discrimination (i.e. no ability to diagnose patients with and without the disease or condition based on the test), 0.7 to 0.8 is considered acceptable, 0.8 to 0.9 is considered excellent, and more than 0.9 is considered outstanding (Mandrekar, Journal of Thoracic Oncology, Volume 5, Number 9, September 2010).

As used herein, the term “balanced accuracy” refers to the average of the sensitivity and specificity of the method. In other words, Balanced Accuracy=(Sensitivity+Specificity)/2.

Due to the nature of AUC and balanced accuracy, the balanced accuracy value of a predictor at a given cut-off will generally be much lower than the AUC value of the predictor. Thus, practitioners in the art would consider a balanced accuracy of 0.6 or above to define that the predictor is acceptable/workable, and a balanced accuracy of 0.75 or above to indicate excellence.

In embodiments, the methods of the invention as described elsewhere herein have an accuracy, balanced accuracy, specificity, sensitivity and/or AUC value of at least 0.6 (60%), 0.65 (65%), 0.7 (70%), 0.75 (75%), 0.8 (80%), 0.85 (85%), 0.9 (90%), 0.91 (91%), 0.92 (92%), 0.93 (93%), 0.94 (94%), 0.95 (95%) or 0.96 (96%).

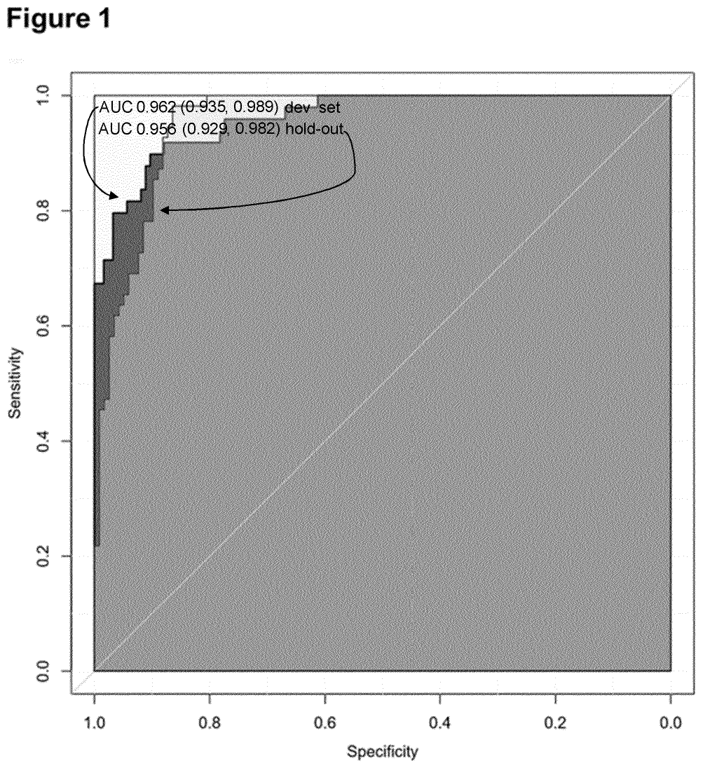

A predictor of the invention using the 121 CpG sites of Table 3, when tested on a hold-out set using a cutoff of p>0.5 for RA-positive, produced excellent results, in particular an overall accuracy of 0.87, a sensitivity of 0.76, a specificity of 0.92, and an AUC value of 0.956 (see Table 1 and FIG. 1).

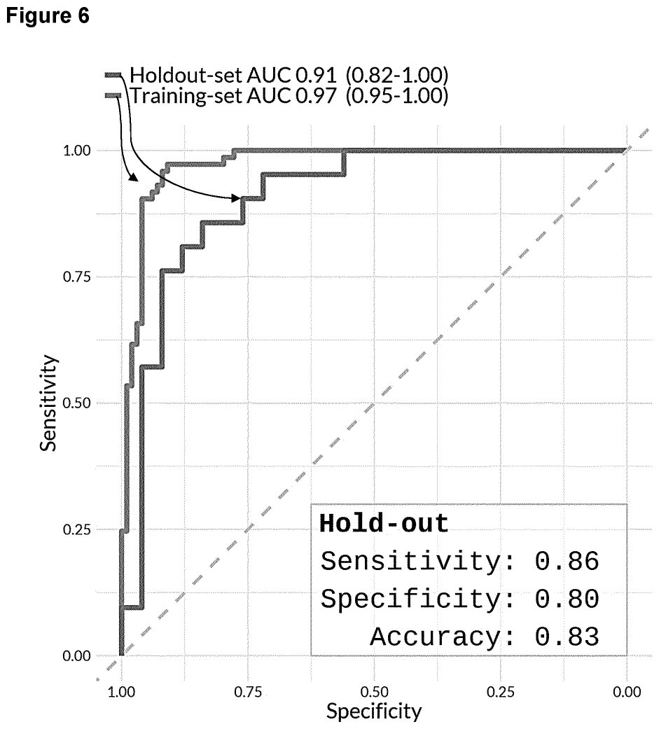

Similarly, a predictor of the invention using the 20 CpG sites of Table 6, when tested on a hold-out set using a cutoff of p>0.44 for RA-positive, produced excellent results, in particular a balanced accuracy of 0.83, a sensitivity of 0.86, a specificity of 0.80, and an AUC value of 0.91 (see Table 8 and FIG. 6).

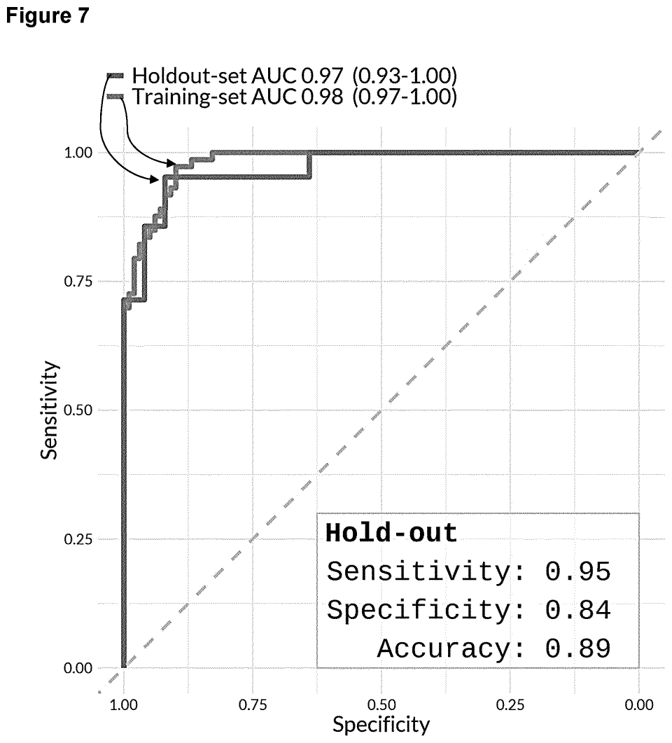

Similarly, a predictor of the invention using the 16 CpG sites of Table 7 plus serology data, when tested on a hold-out set using a cutoff of p>0.36 for RA-positive, produced excellent results, in particular a balanced accuracy of 0.89, a sensitivity of 0.95, a specificity of 0.84, and an AUC value of 0.97 (see Table 8 and FIG. 7).

FIG. 7 shows the AUC of an RA classifier of the invention using methylation levels from the 16 CpG sites listed in Table 7, plus serology data (RF_pos and CCP_pos) as also listed in Table 7). The standardised coefficients used in the classifier/model in respect of each CpG site and in respect of the serology data are also provided in Table 7, together with the intercept used in the classifier/model. The box embedded in the bottom-right of the graph provides the sensitivity, specificity and balanced accuracy values for the predictor (“Accuracy” in the box means “balanced accuracy”).

In embodiments, the method of the invention comprises or further comprises making a diagnosis of rheumatoid arthritis based on the methylation levels referred to elsewhere herein and/or the likelihood referred to elsewhere herein.

The diagnosis may be made on the basis of (or based on) the methylation levels, likelihood value, or readout or result described elsewhere herein. The diagnosis may be considered to be performed by the production of the readout or result itself. Said diagnosis may therefore be computer implemented, e.g. partially or entirely computer implemented, and/or performed in the absence of a clinician. Alternatively or in addition, the diagnosis may be considered to be the conclusion drawn by a clinician based on said methylation levels, likelihood value, or readout or result described elsewhere herein.

In embodiments of any the methods described herein, the method may comprise (or further comprise) delivering a diagnosis. The diagnosis may be based on data used or generated in the method, for example a readout, a result, or methylation levels as described elsewhere herein. The delivering of the diagnosis may be considered to be performed by the production of the readout or result itself. The diagnosis may be delivered in the form of a written or electronic report as described elsewhere herein, or may be delivered orally. The diagnosis may be delivered by a clinician, or by a processing system or computer. The diagnosis may be delivered to any relevant party, for example the subject being tested or an acquaintance thereof, or another clinician.

In embodiments of any of the methods of the invention provided herein, the method may further comprise outputting the data (e.g. readout, result, diagnosis or methylation levels, as the case may be) over a network connection, or displaying the data on a screen, e.g. a computer screen, or on an electronic display.

In embodiments, the subject (e.g. human subject) is a subject at risk of developing rheumatoid arthritis, or is a subject having or suspected of having rheumatoid arthritis, or is a subject that is susceptible to, or believed to be susceptible to, rheumatoid arthritis.

The term “subject” as used herein can also mean “individual”, “patient” or “person”. The methods of the invention as described herein can be carried out on any type of subject which is capable of suffering from rheumatoid arthritis. The methods may be carried out on mammals, for example humans, primates (e.g. monkeys), laboratory mammals (e.g. mice, rats, rabbits, guinea pigs), livestock mammals (e.g. horses, cattle, sheep, pigs) or domestic pets (e.g. cats, dogs).

The subject is preferably a human subject. The subject may be male or female.

The subject may be alive or dead (i.e. the method may be used for post-mortem diagnosis). The human may, for example, be 0-10, 10-20, 20-30, 30-40, 40-50, 50-60, 60-70, 70-80, 80-90, 90-100 or above 100 years old. In embodiments, the subject (e.g. human subject) may be one who is at risk from a particular disease or disorder, e.g. rheumatoid arthritis, or one who has previously suffered from a particular disease or disorder, e.g. rheumatoid arthritis.

Such “at risk”, “suspected” or “susceptible” subjects would be readily identified by a person skilled in the art but would include for example subjects with a family history of rheumatoid arthritis or other autoimmune or autoinflammatory diseases (e.g. as described elsewhere herein), or a genetic predisposition to rheumatoid arthritis or other autoimmune or autoinflammatory diseases, or subjects diagnosed with other autoimmune or autoinflammatory diseases, or subjects with recognized risk factors for rheumatoid arthritis or other autoimmune or autoinflammatory diseases. For example, recognized risk factors for rheumatoid arthritis are being female, having a family history of RA, and exposure to tobacco smoke.

In this way, it can be seen that in some embodiments of the invention, the methods can be carried out on “healthy” patients (subjects) or at least patients (subjects) which are not manifesting any clinical symptoms of rheumatoid arthritis, for example, patients with very early or pre-clinical stage rheumatoid arthritis.

Thus, the methods of the present invention can also be used to monitor disease progression. Such monitoring can take place before, during or after treatment of rheumatoid arthritis by surgery or therapy, e.g. pharmaceutical therapy. Thus, in another aspect the present invention provides a method for monitoring rheumatoid arthritis or monitoring the progression of rheumatoid arthritis in a subject.

Methods of the present invention can be used in the active monitoring of patients which have not been subjected to surgery or therapy, e.g. to monitor the progress of rheumatoid arthritis in untreated patients. For example, serial measurements can allow an assessment of whether or not, or the extent to which, the rheumatoid arthritis is worsening or improving, thus, for example, allowing a more reasoned decision to be made as to whether therapeutic or surgical intervention is necessary or advisable.

As discussed above and elsewhere herein, monitoring can also be carried out, for example, in an individual, e.g. a healthy individual, who is thought to be at risk of developing rheumatoid arthritis or thought to be susceptible to developing rheumatoid arthritis, in order to obtain an early, and ideally pre-clinical, indication of rheumatoid arthritis. The term “monitoring” rheumatoid arthritis as used herein can also be used to mean “monitoring the development of” or “monitoring the progression of” rheumatoid arthritis.

In another aspect, the present invention provides a method for determining the clinical severity of rheumatoid arthritis in a subject. In such methods the methylation level of one or more of the CpG sites as described elsewhere herein in the sample, or the overall likelihood value determined therefrom, shows an association with the severity of the rheumatoid arthritis. Thus, the methylation level of one or more of the CpG sites as described elsewhere herein, or the overall likelihood value determined therefrom, is indicative of the severity of the rheumatoid arthritis. In some embodiments, the more altered (more increased or more decreased as the case may be) the methylation level (or score) of one or more of the CpG sites in comparison to a control level, the greater the likelihood of a more severe form of rheumatoid arthritis. In some embodiments the methods of the invention can thus be used in the selection of patients for therapy.

Serial (periodical) measuring of the methylation level of one or more of the CpG sites in accordance with the present invention and as referred to elsewhere herein may also be used to monitor the severity of RA, looking for either increasing or decreasing levels over time. Observation of altered levels (increase or decrease as the case may be) may also be used to guide and monitor therapy, both in the setting of subclinical disease, i.e. in the situation of “watchful waiting” before treatment or surgery, e.g. before initiation of pharmaceutical therapy or surgery, or during or after treatment to evaluate the effect of treatment and look for signs of therapy failure.

Thus, the present invention also provides a method for predicting the response of a subject to therapy or surgery. For example, a subject with a less severe form or an early stage of rheumatoid arthritis, as determined by the methylation level of one or more of the CpG sites in a sample in accordance with the present invention and as referred to elsewhere herein, is generally more likely to be responsive to therapy or surgery. In such methods the choice of therapy or surgery may be guided by knowledge of the methylation level of one or more of the CpG sites in the sample.

In some embodiments, the invention provides a method of monitoring (e.g. continuously monitoring or performing active surveillance of) a subject having rheumatoid arthritis (e.g. a subject being treated for rheumatoid arthritis). Such monitoring may guide which treatment to use or whether no treatment should be given or whether treatment should be continued or whether the dose of a pharmaceutical agent should be increased or decreased, etc.

In one embodiment, the invention provides the use of the methods of the invention (e.g. screening or diagnostic methods, etc., as described herein) in conjunction with other known screening or diagnostic methods for rheumatoid arthritis, such as magnetic resonance imaging or ultrasound (which can be used to detect joint inflammation and damage), or histological assessment (e.g. synovial tissue biopsy). Thus, for example, the methods of the invention can be used to confirm a diagnosis of rheumatoid arthritis in a subject. In some embodiments the methods of the present invention are used alone.

The methods of the present invention can be carried out on any appropriate biological sample, e.g. any appropriate body fluid sample or tissue sample that contains DNA. In this regard, although blood samples are a common source of DNA, other types of body fluid or tissue sample could be used by a skilled person to extract DNA containing the desired CpG sites, following the teaching as provided herein. Typically the sample has been obtained from (removed from) a subject (e.g. as described elsewhere herein, preferably a human subject). In other aspects, the method further comprises a step of obtaining a sample from the subject.

By obtained from the subject, it is meant that the biological sample is previously obtained, or has been obtained from the subject. Hence, the patient or subject is not required to be present while the methods of the invention are being performed.

Reference herein to “body fluid” includes reference to all fluids derived from the body of a subject. Exemplary fluids include blood (including all blood derived components, for example buffy coat, plasma, serum, etc.), saliva, urine, tears, bronchial secretions or mucus. Preferably, the body fluid is a circulatory fluid (especially blood or a blood component), or urine. Especially preferred body fluids are blood or urine. In some preferred embodiments the sample is a blood sample (e.g. a plasma, serum or buffy coat sample). In some preferred embodiments the sample is a buffy coat sample. In some embodiments the sample is a urine sample. The body fluid or sample may be in the form of a liquid biopsy. The term “sample” also encompasses any material derived by processing a body fluid or tissue sample (e.g. derived by processing a blood or urine sample). Processing of biological samples to obtain a test sample may involve one or more of: digestion, boiling, filtration, distillation, centrifugation, lyophilization, fractionation, extraction, concentration, dilution, purification, inactivation of interfering components, addition of reagents, derivatization, complexation and the like, e.g. as described elsewhere herein.

In embodiments, the biological sample is a blood, saliva, urine, solid tissue (for example cartilage from affected joints), or fecal sample. In preferred embodiments, the biological sample is a blood sample. Preferably, the blood sample is a buffy coat sample or a serum sample or a plasma sample. In embodiments, the sample is a white blood cell (or leukocyte) sample, or is a sample comprising white blood cells (or leukocytes).

Typically, the DNA from the biological sample is genomic DNA.

In embodiments, the method additionally comprises the step of obtaining one or more biological samples from the subject. In some embodiments, one or more of the methylation levels in accordance with the present invention are detected directly in the biological sample, e.g. from within a sample of the subject's blood, blood serum, blood plasma, buffy coat or other sample.

In embodiments, DNA is first isolated and/or purified from the biological sample before the methylation levels are detected. The biological sample may therefore comprise (or consist of, or be) isolated and/or purified DNA.

DNA may be isolated and/or purified from the biological samples by any suitable method which would be well known to a person skilled in the art. Such methods may include cell lysis; treatment with protease, RNase and/or detergent; and DNA purification by ethanol precipitation, phenol-chloroform extraction or minicolumn purification. Specific DNA extraction methods can be used depending on the biological sample in question. For example, where the biological sample is a blood sample, the DNA can be extracted using the Monarch® Protocol for Extraction and Purification of Genomic DNA from Blood (NEB #T3010), or a magnetic bead-based technology such as the ChargeSwitch® gDNA Purification Kit (Thermofisher).

In embodiments, the method of the invention comprises, e.g. further comprises, reporting the results of the method, optionally and conveniently by preparing a written or electronic report.

In embodiments, the method of the invention is implemented by a computer.

In embodiments, the method of the invention comprises, e.g. further comprises, treating said rheumatoid arthritis by therapy or surgery.

There are multiple treatment and therapy options for the management of patients with RA, but the backbone of RA management is conventional synthetic disease-modifying antirheumatic drugs (csDMARDs), especially methotrexate. However, many patients have an inadequate response to or are intolerant of csDMARDs. For such patients, guidelines recommend the addition of either a biologic DMARD (bDMARD) or a targeted synthetic DMARD (tsDMARD), which target specific molecules or molecular structures (targeted therapy). Typically, the first-choice targeted therapy is a tumor necrosis factor inhibitor (TNFi).

Thus, in preferred embodiments the therapy comprises a step of administering to the subject a therapeutically effective amount of one or more agents selected from the group consisting of synthetic disease-modifying antirheumatic drugs (csDMARDs), preferably methotrexate, a biologic DMARD (bDMARD), a targeted synthetic DMARD, or a tumor necrosis factor inhibitor (TNFi).

Subjects with rheumatoid arthritis may elect to have surgery to reduce joint pain and improve everyday function. The most common surgeries are joint replacement, arthrodesis and synovectomy. In the case of joint replacement, patients may elect to have joint replacements for large joints such as shoulders, hips, or knees, and/or smaller joints in the fingers and toes. Joint replacement surgery may involve removing all or part of a damaged joint, and inserting a synthetic replacement.

Thus, preferably the surgery is joint replacement, arthrodesis or synovectomy. The joint replacement is preferably shoulder, hip, knee, finger joint or toe joint replacement. The joint replacement comprises full or partial joint replacement. The joint replacement may comprise inserting a synthetic replacement.

In embodiments, the method of the invention comprises, e.g. further comprises, altering, ceasing or continuing treatment of said subject.

In embodiments, the method of the invention comprises, e.g. further comprises, a step of measuring the methylation levels before the step of using the methylation levels.

In embodiments, the method of the invention comprises, e.g. further comprises, providing DNA (said DNA) from a biological sample obtained from the subject before the step of measuring the methylation levels.

In one or more embodiments, a method of the invention is provided comprising a first step of extracting DNA (e.g. genomic DNA) from a sample, e.g. a biological sample. In a second step, the DNA methylation levels at multiple CpG sites as defined elsewhere herein are measured. Each measurement measures the extent of methylation at a particular CpG site.

In another aspect, the invention provides a computer program, software, or computer readable storage medium (e.g. a non-transitory and/or tangible computer readable storage medium), comprising instructions that, when executed by a processing system, cause the processing system to process data representative of methylation levels of at least or at most:
1, 4, 5, 10, 11, 15, 20, 23, 25, 30, 40, 50, 60, 70, 80, 90, 100, 110, 120, 121, 130, 140 or 145 CpG sites selected from the list of CpG site numbers 1 to 145 of Table 9;

In another aspect, the invention provides a computer program comprising instructions that, when executed by a processing system, cause the processing system to process data representative of methylation levels of at least or at most:
1, 4, 5, 10, 11, 15, 20, 25, 30, 31, 40, 50, 60, 70, 80, 90, 100, 110, 120 or 121 CpG sites selected from the list of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from a subject, to calculate a likelihood of the subject having rheumatoid arthritis.

In another aspect, the invention provides software comprising instructions that, when executed by a processing system, cause the processing system to process data representative of methylation levels of at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from a subject, to calculate a likelihood of the subject having rheumatoid arthritis.

The software or computer program may be stored on a non-transitory and/or tangible computer-readable storage medium, such as a hard-drive, a CD-ROM, a solid-state memory, etc., or may be communicated by a transitory signal such as data over a network.

Hence, in another aspect, the invention provides a computer readable storage medium, e.g. a non-transitory and/or tangible computer readable storage medium, comprising instructions that, when executed by a processing system, cause the processing system to process data representative of methylation levels of at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from a subject, to calculate a likelihood of the subject having rheumatoid arthritis.

These embodiments provide a means for executing or implementing the methods of the invention as described herein. Thus, in these embodiments, any other number of the CpG sites or other features as described elsewhere herein for the methods of the invention can be used.

In alternative embodiments, the instructions cause the processing system to calculate the likelihood as a non-linear function of the combination of said methylation levels in accordance with the invention as described elsewhere herein.

In embodiments, the instructions cause the processing system to calculate the likelihood as a function of a linear combination of said methylation levels.

In embodiments, the linear combination of said methylation levels comprises a weighted sum of said methylation levels.

In embodiments, the instructions cause the processing system to calculate the likelihood as a logistic function of a linear combination of said methylation levels.

In embodiments, the instructions cause the processing system to receive data representative of said methylation levels and input the data to an algorithm for evaluating said function to determine the likelihood of the subject having rheumatoid arthritis.

In embodiments, the computer program, software, or non-transitory (or tangible) computer readable storage medium comprises computer-readable code that, when executed by a processing system (or a computer), causes the processing system (or the computer) to perform one or more additional operations comprising: sending information corresponding to the methylation levels of the set of CpG sites in the biological sample to a tangible data storage device.

The methods disclosed herein may be fully or wholly computer-implemented methods. Alternatively, the methods disclosed herein may be partially computer-implemented methods. Any of the method steps disclosed herein may, wherever appropriate, be implemented as steps of the method, using any appropriate hardware and/or software. The computer software disclosed herein may be on a transitory or a non-transitory computer-readable medium. The diagnostic algorithm could be implemented on one or more further computer processing systems that are distinct from the computer processing system that is configured to train the model.

The invention may also be provided in a fully developed software package or web-based program. For example, a user may access a webpage and upload their DNA methylation data.

The program then emails the results, including the indication of the presence or absence of rheumatoid arthritis, to the user.

Where “processing system” is recited, it should be understood that “computer” or “computer system” is also contemplated alternatively or in addition.

In another aspect, the invention provides a processing system configured to perform the method of the invention.

In another aspect, the invention provides a processing system (or a computer or computer system) configured to run the algorithm or software of the invention as provided elsewhere herein or configured to perform the methods of the invention.

In another aspect, the invention provides a method of screening or diagnosing, etc., rheumatoid arthritis in a subject, the method comprising calculating, optionally implemented by a computer, a likelihood (or probability) of the subject having rheumatoid arthritis using measurements of methylation levels of at least or at most:
1, 4, 5, 10, 11, 15, 20, 23, 25, 30, 40, 50, 60, 70, 80, 90, 100, 110, 120, 121, 130, 140 or 145 CpG sites selected from the list of CpG site numbers 1 to 145 of Table 9;
obtained from a DNA sample of the subject. In alternative embodiments, any other number of the CpG sites or other features as described herein for the methods of the invention can be used.

In another aspect, the invention provides a method of monitoring rheumatoid arthritis in a subject, the method comprising:
(a) using the methylation levels of at least or at most: 1, 4, 5, 10, 11, 15, 20, 23, 25, 30, 40, 50, 60, 70, 80, 90, 100, 110, 120, 121, 130, 140 or 145 CpG sites selected from the list of CpG site numbers 1 to 145 of Table 9; in DNA from a biological sample obtained from the subject at a first time point; and
(b) comparing said methylation levels to the methylation levels of the same at least or at most: 1, 4, 5, 10, 11, 15, 20, 23, 25, 30, 40, 50, 60, 70, 80, 90, 100, 110, 120, 121, 130, 140 or 145 CpG sites selected from the list of CpG site numbers 1 to 145 of Table 9; in DNA from a biological sample obtained from the subject at a second time point. In alternative embodiments, any other number of the CpG sites or other features as described herein for the methods of the invention can be used.

In another aspect, the invention provides a method of obtaining an indication of the efficacy of a drug which is being used to treat rheumatoid arthritis in a subject, the method comprising:
(a) using the methylation levels of at least or at most: 1, 4, 5, 10, 11, 15, 20, 23, 25, 30, 40, 50, 60, 70, 80, 90, 100, 110, 120, 121, 130, 140 or 145 CpG sites selected from the list of CpG site numbers 1 to 145 of Table 9; in DNA from a biological sample obtained from the subject at a first time point; and
(b) comparing said methylation levels to the methylation levels of the same at least or at most: 1, 4, 5, 10, 11, 15, 20, 23, 25, 30, 40, 50, 60, 70, 80, 90, 100, 110, 120, 121, 130, 140 or 145 CpG sites selected from the list of CpG site numbers 1 to 145 of Table 9; within a biological sample obtained from the subject at a second time point, wherein a drug has been administered to the subject in the interval between the first and second time points or has been administered at any other appropriate time point, for example at or around the same time as the first time point, for example at a time point where a base-line level of methylation can be measured. In alternative embodiments, any other number of the CpG sites or other features as described herein for the methods of the invention can be used.

In another aspect, the invention provides a method of screening or diagnosing, etc., rheumatoid arthritis in a subject, the method comprising calculating, optionally implemented by a computer, a likelihood (or probability) of the subject having rheumatoid arthritis using measurements of methylation levels of at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
obtained from a DNA sample of the subject. In alternative embodiments, any other number of the CpG sites or other features as described herein for the methods of the invention can be used.

In another aspect, the invention provides a method of monitoring rheumatoid arthritis in a subject, the method comprising:
(a) using the methylation levels of at least or at most: 1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3 (or any other number of the CpG sites as described elsewhere herein); and/or 1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5; in DNA from a biological sample obtained from the subject at a first time point; and
(b) comparing said methylation levels to the methylation levels of the same at least or at most: 1, 4, 5, 10, 11, 15, 20, 25, 30, 31, 40, 50, 60, 70, 80, 90, 100, 110, 120 or 121 CpG sites of Table 3 (or the same any other number of the CpG sites as described elsewhere herein); and/or 1, 2, 3, 4, 5, 6, 7, 8, 9, 10, 11, 12, 13, 14, 15, 16, 17, 18, 19, 20, 21, 22, 23 or 24 CpG sites of Table 5; in DNA from a biological sample obtained from the subject at a second time point.

In another aspect, the invention provides a method of obtaining an indication of the efficacy of a drug which is being used to treat rheumatoid arthritis in a subject, the method comprising:
(a) using the methylation levels of at least or at most: 1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3 (or any other number of the CpG sites as described elsewhere herein); and/or 1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5; in DNA from a biological sample obtained from the subject at a first time point; and
(b) comparing said methylation levels to the methylation levels of the same at least or at most: 1, 4, 5, 10, 11, 15, 20, 25, 30, 31, 40, 50, 60, 70, 80, 90, 100, 110, 120 or 121 CpG sites (or the same any other number of the CpG sites as described elsewhere herein); and/or 1, 2, 3, 4, 5, 6, 7, 8, 9, 10, 11, 12, 13, 14, 15, 16, 17, 18, 19, 20, 21, 22, 23 or 24 CpG sites of Table 5; within a biological sample obtained from the subject at a second time point, wherein a drug has been administered to the subject in the interval between the first and second time points or has been administered at any other appropriate time point, for example at or around the same time as the first time point, for example at a time point where a base-line level of methylation can be measured.

The biological samples obtained in steps (a) and (b) should be directly comparable, e.g. the biological samples must both be of the same type (e.g. both are blood samples) and subsequently treated in the same manner. The first time point may, for example, be at an early stage of the rheumatoid arthritis. The second time point may be at a later stage of the rheumatoid arthritis, or after the subject has been treated with medicament suitable for the treatment of rheumatoid arthritis. The first and second time points may be any suitable time intervals, e.g. at least one week apart, 1-12 months apart, or at least 1, 2, 3, 4 or 5 years apart. Serial (periodic) measuring of the level of the methylation levels of one or more of the CpG sites in accordance with the present invention may also be used for disease monitoring, e.g. assessing disease severity, looking for either increasing or decreasing levels (or scores or likelihoods or likelihood values) over time. In some embodiments, an altering methylation level or score or likelihood (increase or decrease, as appropriate) of one or more of the CpG sites in accordance with the present invention over time (e.g. in comparison to a control level or base-line or earlier level in the same subject, e.g. a level moving further away from the control level, base-line or earlier level in the same subject) may indicate a worsening disease state, severity or prognosis. In some embodiments, an altering level (increase or decrease, as appropriate) of the methylation level of one or more of the CpG sites in accordance with the present invention over time (e.g. in comparison to a control level, e.g. a level moving closer to the control level) may indicate an improving disease state, severity or prognosis.

In embodiments, a change in the methylation levels between the first and second time points in any aspects referred to herein is indicative of a change in severity of rheumatoid arthritis in the subject.

In another aspect, the invention provides a method of treating rheumatoid arthritis in a subject, the method comprising:
(a) obtaining an indication of the presence of rheumatoid arthritis in a subject by performing a method of the present invention as described elsewhere herein; and
(b) administering a treatment appropriate for treating rheumatoid arthritis to the subject if an indication of the presence of rheumatoid arthritis in the subject is obtained, thereby treating rheumatoid arthritis in the subject.

In another aspect, the invention provides a method of preventing rheumatoid arthritis in a subject, the method comprising:
(a) obtaining an indication of an increased risk of rheumatoid arthritis in a subject (e.g. a healthy subject or an at risk or susceptible subject) by performing a method of the present invention as described elsewhere herein; and
(b) administering a treatment appropriate for preventing rheumatoid arthritis in the subject if an indication of an increased risk of rheumatoid arthritis in the subject is obtained, thereby preventing rheumatoid arthritis in the subject.

In another aspect, the invention provides a method of treating rheumatoid arthritis in a subject, the method comprising the step of:
(a) administering a treatment appropriate for treating rheumatoid arthritis to the subject, wherein, prior to administration, an indication of the presence of rheumatoid arthritis in the subject, has been obtained by a method of the invention.

In the above methods, the treatment to be administered can also be a surgical treatment, e.g. as described elsewhere herein.

A number of different methods for detecting methylation levels of CpG sites are known and described in the literature and any of these may be used according to the present invention. At its simplest, the methylation level or state of a CpG site may be detected by hybridisation to a probe (e.g. an oligonucleotide probe) and many such hybridisation protocols have been described (see e.g. Sambrook et al., Molecular cloning: A Laboratory Manual, 3rd Ed., 2001, Cold Spring Harbor Press, Cold Spring Harbor, NY). Typically, the detection will involve a hybridisation step and/or an in vitro amplification step.

In one embodiment, the target nucleic acid, e.g. the methylated or unmethylated form of a particular CpG site, in a sample, may be detected by using an oligonucleotide with a label attached thereto, which can hybridise to the nucleic acid sequence of interest. Such a labelled oligonucleotide will allow detection by direct means or indirect means. In other words, such an oligonucleotide may be used simply as a conventional oligonucleotide probe. After contact of such a probe with the sample under conditions which allow hybridisation, and typically following a step (or steps) to remove unbound labelled oligonucleotide and/or non-specifically bound oligonucleotide, the signal from the label of the probe emanating from the sample may be detected. In preferred embodiments the label is selected such that it is detectable only when the probe is hybridised to its target.

The probe may have a nucleic acid sequence complementary to the sequence of the CpG site of interest or a derivative thereof. The probe may be complementary to the CpG site (i.e. the dinucleotide “CG” sequence) and certain adjacent residues.

The probe may alternatively be complementary to a derivative of the CpG site and certain immediately adjacent residues, for example 10, 20, 30, 40, 50 or 60 immediately adjacent residues. The immediately adjacent residues may for example be residues to the 3′ side of (downstream of) the CG dinucleotide. This probe design format is known in the art.

CpG methylation can be detected using two different types of probe as used in the Illumina Infinium I Methylation Assay. The probes may each be linked to a solid support, for example a bead. The first probe type (named the U type in the Infinium I assay) has the sequence “CA” at its 3′ end, and thus is complementary to the sequence of an unmethylated CpG site which has been bisulfite treated (i.e. to “UG”) and subsequently amplified (i.e. “TG”). The second probe type (named the M type in the Infinium I assay) has the sequence “CG” at its 3′ end, and thus is complementary to the sequence of a methylated CpG site, whether bisulfite-treated or not (i.e. “CG”). The probes may be complementary to said CpG sites and certain immediately adjacent residues (or a derivative of said sequence). The immediately adjacent residues may for example be residues to the 3′ side of (downstream of) the CG dinucleotide. Annealing of complementary probes to their target sites enables single-nucleotide (or single-base) extension. In order to enable detection, the nucleotide incorporated in the single-nucleotide extension may be labelled with an appropriate fluorophore (which indicates methylation or non-methylation), and the fluorescent signal may be detected using an imaging apparatus, for example Illumina iScan.

Alternatively or in addition, CpG methylation may be detected using a single type of probe as used in the Infinium II Methylation Assay. The probe may have at its 3′ end a cytosine residue suitable for hybridising to the guanine of the “CG” sequence. The probe may be complementary to said guanine and certain immediately adjacent residues (or a derivative of said sequence). The immediately adjacent residues may for example be residues to the 3′ side of (downstream of) the CG dinucleotide. The probe may be linked to a solid support, for example a bead. The probe can therefore target or hybridise to the CpG site irrespective of the sequence of the CpG site after bisulfite treatment. After hybridisation to the bisulfite treated sequence of interest, single-base extension is conducted to identify the second nucleotide of the bisulfite-treated CpG site, and thus whether the CpG site was methylated or unmethylated. In order to enable detection, the nucleotide incorporated in the single-nucleotide extension may be labelled with an appropriate fluorophore (which indicates methylation or non-methylation), and the fluorescent signal may be detected using an imaging apparatus, for example Illumina iScan.

For detecting (or measuring) the methylation level of CpG sites which, when methylated, are methylated on both strands (i.e. the cytosine of the CpG site is methylated on both the sense and antisense DNA strands), the probe or probes can be designed to be targeted to the sequence of the sense strand of the CpG site, or the antisense strand of the CpG site. For CpG sites which, when methylated, are hemimethylated (i.e. the cytosine of the CpG site on one strand is methylated (e.g. the sense strand), while cytosine of the CpG site on the other strand is unmethylated (e.g. the antisense strand)), the probe or probes can be designed to be targeted to the sequence of the strand on which methylation occurs. Hence, probes for use in accordance with the invention may be targeted towards (or complementary to) the sense strand of a CpG site or the antisense strand of a CpG site.

The term “probe” as used herein refers to an oligonucleotide capable of binding in a base-specific manner to a complementary strand of nucleic acid. The term “probe” as used herein can also refer to a surface-immobilized molecule that can be recognized by a particular target as well as molecules that are not immobilized and are coupled to a detectable label. The terms “probe” and “primer” can be used interchangeably herein. The probe is conveniently a nucleic acid probe and thus can be a DNA or RNA oligonucleotide, typically a DNA oligonucleotide. The probe may be for example 10, 20, 30, 40, 50, 60 or 70 nucleotides in length.

The term “complementary” or “targeted” as used herein can refer to the hybridization or base pairing between nucleotides or nucleic acids (e.g. between probes), such as, for instance, between the two strands of a double stranded DNA molecule or between an oligonucleotide primer or probe and a primer or probe binding site on a single stranded nucleic acid to be sequenced or amplified.

In another embodiment, the target CpG site in a sample may be detected or identified by using an oligonucleotide probe which is labelled only when hybridised to its target sequence, i.e. the probe may be selectively labelled. Conveniently, selective labelling may be achieved using labelled nucleotides, i.e. by incorporation into the oligonucleotide probe of a nucleotide carrying a label. In other words, selective labelling may occur by chain extension of the oligonucleotide probe using a polymerase enzyme which incorporates a labelled nucleotide, preferably a labelled dideoxynucleotide (e.g. ddATP, ddCTP, ddGTP, ddTTP, ddUTP). This approach to the detection of specific nucleotide sequences is sometimes referred to as primer extension analysis. Suitable primer extension analysis techniques are well known to the skilled person, e.g. those techniques disclosed in WO99/50448, the contents of which are incorporated herein by reference.

Modifications of the basic PCR method such as qPCR (Real Time PCR) have been developed that can provide quantitative information on the template being amplified. Numerous approaches have been taken although the two most common techniques use double-stranded DNA binding fluorescent dyes or selective fluorescent reporter probes.

Fluorescent reporter probes used in qPCR may be sequence specific oligonucleotides, typically RNA or DNA, that have a fluorescent reporter molecule at one end and a quencher molecule at the other (e.g. the reporter molecule is at the 5′ end and a quencher molecule at the 3′ end or vice versa). The probe is designed so that the reporter is quenched by the quencher. The probe is also designed to hybridise selectively to particular regions of complementary sequence which might be in the template. If these regions are between the annealed PCR primers the polymerase, if it has exonuclease activity, will degrade (depolymerise) the bound probe as it extends the nascent nucleic acid chain it is polymerising. This will relieve the quenching and fluorescence will rise. Accordingly, by measuring fluorescence after every PCR cycle, the relative amount of amplification product can be monitored in real time. Through the use of internal standard and controls, this information can be translated into quantitative data.

The amplification product may be detected, and amounts (levels) of amplification product can be determined by any convenient means. A vast number of techniques are routinely employed as standard laboratory techniques and the literature has descriptions of more specialised approaches. At its most simple the amplification product may be detected by visual inspection of the reaction mixture at the end of the reaction or at a desired time point. Typically the amplification product will be resolved with the aid of a label that may be preferentially bound to the amplification product. Typically a dye substance, e.g. a colorimetric, chromomeric fluorescent or luminescent dye (for instance ethidium bromide or SYBR green) is used. In other embodiments a labelled oligonucleotide probe that preferentially binds the amplification product is used.

In some embodiments, the relative abundance of the methylated or unmethylated CpG site in association with (e.g. physical association with or in complex with) the probe is determined. Thus, in some embodiments the level of a complex of the methylated or unmethylated CpG site and the probe used to detect the methylated or unmethylated CpG site is determined. In some embodiments the level of a methylated or unmethylated CpG site in association with (e.g. in complex with) a primer (or extended primer) or probe (e.g fluorescent reporter probe) or dye or the like may be determined.

DNA methylation of the CpG sites can be measured using various approaches, which range from commercial array platforms (e.g. from Illumina™) to sequencing approaches of individual genes. This includes standard lab techniques or array platforms. For a review of some methylation detection methods, see, Oakeley, E. J., Pharmacology & Therapeutics 84:389-400 (1999).

Available methods of the measuring the DNA methylation levels of CpG sites include, but are not limited to: methylation-sensitive sequencing, a microarray-based method, (e.g. using an Illumina microarray such as an Illumina 450 k array or Illumina Infinium Methylation EPIC Kit), reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), combined bisulphate-restriction analysis, methylation sensitive single nucleotide probe extension, methylation-sensitive single-strand conformation analysis (MS-SSCA), high resolution melting analysis (HRM), methylation-sensitive single-nucleotide primer extension (MS-SnuPE), base-specific cleavage/MALDI-TOF, Combined Bisulfite Restriction Analysis (COBRA), methylated DNA immunoprecipitation (MeDIP), pyrosequencing or bisulfite sequencing. For example, measuring a methylation level can comprise performing array-based PCR (e.g., digital PCR), targeted multiplex PCR, or direct sequencing without bisulfite treatment (e.g., via a nanopore technology). In some aspects, determining methylation status comprises methylation specific PCR, real-time methylation specific PCR, quantitative methylation specific PCR (QMSP), or bisulfite sequencing. In certain aspects, a method according to the embodiments comprises treating DNA in or from a sample with bisulfite (e.g., sodium bisulfite) to convert unmethylated cytosines of CpG dinucleotides to uracil.

In more detail, the following assays can also be used to measure DNA methylation levels:
a) Molecular break light assay for DNA adenine methyltransferase activity is an assay that is based on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase.
b) Methylation-Specific Polymerase Chain Reaction (PCR) is based on a chemical reaction of sodium bisulfite with DNA that converts unmethylated cytosines of CpG dinucleotides to uracil or UpG, followed by traditional PCR. However, methylated cytosines will not be converted in this process, and thus probes are designed to overlap the CpG site of interest, which allows one to determine methylation status as methylated or unmethylated. The beta value can be calculated as the proportion of methylation.
c) Whole genome bisulfite sequencing, also known as BS-Seq, is a genome-wide analysis of DNA methylation. It is based on the sodium bisulfite conversion of genomic DNA, which is then sequenced on a Next-Generation Sequencing (NGS) platform. The sequences obtained are then re-aligned to the reference genome to determine methylation states of CpG dinucleotides based on mismatches resulting from the conversion of unmethylated cytosines into uracil.
d) The HpaII tiny fragment Enrichment by Ligation-mediated PCR (HELP) assay is based on restriction enzymes' differential ability to recognize and cleave methylated and unmethylated CpG DNA sites.
e) Methyl Sensitive Southern Blotting is similar to the HELP assay but uses Southern blotting techniques to probe gene-specific differences in methylation using restriction digests. This technique is used to evaluate local methylation near the binding site for the probe.
f) ChIP-on-chip assay is based on the ability of commercially prepared antibodies to bind to DNA methylation-associated proteins like MeCP2.
g) Restriction landmark genomic scanning is a complicated and now rarely-used assay is based upon restriction enzymes' differential recognition of methylated and unmethylated CpG sites. This assay is similar in concept to the HELP assay.
h) Methylated DNA immunoprecipitation (MeDIP) is analogous to chromatin immunoprecipitation. Immunoprecipitation is used to isolate methylated DNA fragments for input into DNA detection methods such as DNA microarrays (MeDIP-chip) or DNA sequencing (MeDIP-seq).
i) Pyrosequencing of bisulfite treated DNA is a sequencing of an amplicon made by a normal forward primer (or probe) but a biatenylated reverse primer (or probe) to PCR the gene of choice. The Pyrosequencer then analyses the sample by denaturing the DNA and adding one nucleotide at a time to the mix according to a sequence given by the user. If there is a mismatch, it is recorded and the percentage of DNA for which the mismatch is present is noted. This gives the user a percentage methylation per CpG island.

In certain embodiments of the invention, the DNA (e.g. genomic DNA) is hybridized to a complementary sequence (e.g. a synthetic polynucleotide sequence) that is coupled to a matrix (e.g. one disposed within a microarray). Optionally, the DNA (e.g. genomic DNA) is transformed from its natural state via amplification by a polymerase chain reaction process. For example, prior to or concurrent with hybridization to an array, the sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al, Academic Press, San Diego, Calif, 1990); Mattila et al, Nucleic Acids Res. 19, 4967 (1991); Eckert et al, PCR Methods and Applications 1, 17 (1991); PCR (Eds. McPherson et al, IRL Press, Oxford). The sample may be amplified on the array.

Any appropriate statistical approach can be used to relate the methylation levels to an indication of the presence or absence of rheumatoid arthritis, e.g. a weighted sum of the methylation levels can be applied to a logistic function as described herein. Using conventional regression model/analysis tools and methodologies known in the art, a number of diagnostic prediction models are contemplated for use with specific DNA samples (e.g. genomic DNA samples) and/or specific analysis techniques and/or specific individual populations.

In embodiments, a logistic regression model may predict the presence or absence of rheumatoid arthritis based on a weighted sum of the methylation levels optionally plus an offset (or regression intercept). To identify the weights for the weighted sum, one can use the regression coefficients of a regression model.

The coefficient values (weights) can be tailored to the subject being analysed. For example, if a model is applied to female patients only, then one set of coefficients can be used. Alternatively, if a model is applied exclusively to smokers, another set of coefficients can be used. Alternatively, coefficients can be fixed, for example, when a model is broadly applied to a heterogeneous group of subjects, e.g. the selection of weights provided in Table 3.

Coefficient values (weights) in various models can also reflect the specific assay that is used to measure the methylation levels. Different machines may give different methylation values, which are closer or farther away from the true methylation values. The coefficients may change when the model is re-trained for another machine. For example, for beta values measured on Illumina™ methylation microarray platforms there can be one set of coefficients (weights), while for other methylation measures (e.g. using sequencing technology) there can be another set of coefficients (weights) etc. Other values may also be used instead, such as M values (transformed versions of beta values). The methylation levels measured by the technique are preferably measured using an Illumina 450 k array or Illumina Infinium Methylation EPIC Kit, or an array of similar quality.

In addition to using art accepted modeling techniques (e.g. regression analyses), embodiments of the invention can include a variety of art accepted technical processes. For example, in certain embodiments of the invention, a bisulfite conversion process is performed so that cytosine residues in the DNA (e.g. genomic DNA) are transformed to uracil, while 5-methylcytosine residues in the DNA (e.g. genomic DNA) are not transformed to uracil. Kits for DNA bisulfite modification are commercially available from, for example, MethylEasy™ (Human Genetic Signatures™) and CpGenome™ Modification Kit (Chemicon™). See also, WO04096825A1, which describes bisulfite modification methods and Olek et al. Nuc. Acids Res. 24:5064-6 (1994), which discloses methods of performing bisulfite treatment and subsequent amplification. Bisulfite treatment allows the methylation status of cytosines to be detected by a variety of methods. For example, any method that may be used to detect a SNP may be used, for examples, see Syvanen, Nature Rev. Gen. 2:930-942 (2001). Methods such as single base extension (SBE) may be used or hybridization of sequence specific probes similar to allele specific hybridization methods. In another aspect the Molecular Inversion Probe (MIP) assay may be used.

In another aspect, the invention provides a kit for screening for rheumatoid arthritis in a subject, said kit comprising: (i) probes (or other appropriate entities); (ii) an array of probes (or other appropriate entities); or (iii) a solid support (e.g. a chip) comprising probes (or other appropriate entities; for detecting (or measuring) the methylation levels of at least or at most:
1, 4, 5, 10, 11, 15, 20, 23, 25, 30, 40, 50, 60, 70, 80, 90, 100, 110, 120, 121, 130, 140 or 145 CpG sites selected from the list of CpG site numbers 1 to 145 of Table 9;
   in DNA from a biological sample obtained from the subject.

In another aspect, the invention provides a kit for screening for rheumatoid arthritis in a subject, said kit comprising probes (or other appropriate entities) for detecting (or measuring) the methylation levels of at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5:
   in DNA from a biological sample obtained from the subject.

Alternatively viewed, the invention provides a kit for screening for rheumatoid arthritis, said kit comprising an array of probes (or other appropriate entities) for detecting (or measuring) the methylation or methylation level of a selection of CpG sites, wherein the selection of CpG sites consists of at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from the subject.

Alternatively viewed, the invention provides a kit for screening for rheumatoid arthritis, said kit comprising a solid support (e.g. a chip) comprising probes (or other appropriate entities) for (or capable of) detecting (or measuring) the methylation or methylation level of a CpG site of at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from the subject.

In one embodiment, the kit is used to determine (or the kit is suitable for determining) whether or not a subject has rheumatoid arthritis by utilizing measurements of methylation levels at specific CpG sites in cells derived from the biological sample, for example blood or saliva. Microfluidics devices can be applied to easily accessible tissues/fluids such as blood, buccal cells, or saliva. Optionally, the kit comprises a plurality of probes for amplifying DNA sequences (e.g. genomic DNA sequences) of the CpG sites (or bisulfite-treated forms of the CpG sites) in accordance with the invention as described elsewhere herein. Optionally, the kit comprises bisulfite or sodium bisulfite.

In embodiments, a kit is provided for obtaining information useful to determine the presence or absence of rheumatoid arthritis in a subject, the kit comprising a plurality of probes (or other appropriate entities) specific for (or specifically targeted to) at least or at most:
1, 4, 5, 10, 11, 15, 20, 23, 25, 30, 40, 50, 60, 70, 80, 90, 100, 110, 120, 121, 130, 140 or 145 CpG sites selected from the list of CpG site numbers 1 to 145 of Table 9; in DNA from a biological sample obtained from the subject.

In embodiments, a kit is provided for obtaining information useful to determine the presence or absence of rheumatoid arthritis in a subject, the kit comprising a plurality of probes (or other appropriate entities) specific for (or specifically targeted to) at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or
1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   in DNA from a biological sample obtained from the subject.

In embodiments of the kits as described herein, the probes (or other appropriate entities) are for detecting (or measuring) the methylation levels of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 CpG sites selected from the list in Table 3.

In embodiments of the kits as described herein, the probes (or other appropriate entities) are for detecting (or measuring) the methylation levels of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 CpG sites selected from CpG sites 1 to 31 in Table 3. In preferred embodiments, the kit comprises probes for detecting (or measuring) the methylation levels of at least or at most 1, 5, 10, 15, 20, 25, 30 or 31 CpG sites selected from CpG site numbers 1 to 31 of Table 3.

In embodiments of the kits as described herein, the probes (or other appropriate entities) are for detecting (or measuring) the methylation levels of at least or at most:
1, 2, 3, 4, 5, 6, 7, 8, 9, 10, 11, 12, 13, 14, 15, 16, 17, 18, 19 or 20 CpG sites selected from the list of CpG site numbers 1 to 20 of Table 6; and/or
   1, 2, 3, 4, 5, 6, 7, 8, 9, 10, 11, 12, 13, 14, 15 or 16 CpG sites selected from the list of CpG site numbers 1 to 16 of Table 7.

In embodiments, the kit is (or comprises) an array or microarray, or is in the form of an array or microarray. The term “array” or “microarray” as used herein refers to an intentionally created collection of molecules (e.g. probes or other appropriate entities) which can be prepared either synthetically or biosynthetically (e.g. Illumina™ HumanMethylation27 microarrays). The array can assume a variety of formats, for example, libraries of probes for targeting the desired CpG site sequences; or libraries of probes for targeting the desired CpG site sequences tethered to resin beads, silica chips, or other solid supports. DNA methylation microarrays commonly comprise tethered nucleic acid probes, for example the Illumina Infinium® HumanMethylation450 BeadChip.

However, the kits of the invention as described herein are specifically designed for the detection (or measurement) of the CpG sites of the invention as described elsewhere herein. In other words, said kits are for use in, or in accordance with, the methods of the invention as described elsewhere herein.

Thus, the probe (or other appropriate entity) component of said kits generally comprises (or consists of) a relatively small subset of probes (or other appropriate entities), e.g. a subset of probes for detecting (or measuring) at least or at most:
1, 4, 5, 10, 15, 20, 23, 25, 30, 40, 50, 60, 70, 80, 90, 100, 110, 120, 121, 130, 140 or 145 CpG sites selected from the list of CpG site numbers 1 to 145 of Table 9; but generally not exceeding more than or up to 145, 150, 160, 170, 180, 190, 200, 300, 400 or 500 different probes in total.

Thus, in other embodiments the present invention provides a kit for screening for rheumatoid arthritis in a subject, said kit comprising probes for detecting the methylation levels of at least or at most:
1, 4, 5, 10, 15, 20, 23, 25, 30, 40, 50, 60, 70, 80, 90, 100, 110, 120, 121, 130, 140 or 145 CpG sites selected from the list of CpG site numbers 1 to 145 of Table 9;
   in DNA from a biological sample obtained from the subject, optionally wherein the probe (or CpG probe) component of the kit consists of up to 145, 150, 160, 170, 180, 190, 200, 300, 400 or 500 different probes or consists of probes for detecting the methylation levels of up to 145, 150, 160, 170, 180, 190, 200, 300, 400 or 500 CpG sites.

More preferably, the probe (or other appropriate entity) component of said kits generally comprises (or consists of) a relatively small subset of probes (or other appropriate entities), e.g. a subset of probes for detecting (or measuring) at least or at most:
1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or 1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5;
   but generally not exceeding more than or up to 24, 30, 40, 50, 60, 70, 80, 90, 100, 110, 120, 121, 130, 140, 150, 160, 170, 180, 190, 200, 300, 400 or 500 different probes in total.

Thus, in other embodiments the present invention provides a kit for screening for rheumatoid arthritis in a subject, said kit comprising probes for detecting the methylation levels of at least or at most: 1, 4, 5, 10, 11, 15, 20, 25, 30, 31, 40, 50, 60, 70, 80, 90, 100, 110, 120 or 121 CpG sites selected from the list of CpG site numbers 1 to 121 of Table 3; and/or 1, 2, 3, 4, 5, 6, 7, 8, 9, 10, 11, 12, 13, 14, 15, 16, 17, 18, 19, 20, 21, 22, 23 or 24 CpG sites selected from the list of CpG site numbers 1 to 24 of Table 5; in DNA from a biological sample obtained from the subject, optionally wherein the probe (or CpG probe) component of the kit consists of up to 24, 30, 40, 50, 60, 70, 80, 90, 100, 110, 120, 121, 130, 140, 150, 160, 170, 180, 190, 200, 300, 400 or 500 different probes or consists of probes for detecting the methylation levels of up to 24, 30, 40, 50, 60, 70, 80, 90, 100, 110, 120, 121, 130, 140, 150, 160, 170, 180, 190, 200, 300, 400 or 500 CpG sites.

Exemplary kits may for example only comprise probes (or other appropriate entities) for detecting (or measuring) up to the 145 CpG sites of the invention (i.e. the 145 CpG sites of Table 9), or up to or only the 121 CpG sites of Table 3, or up to or only the 24 CpG sites of Table 5, or up to or only the 20 CpG sites of Table 6, or up to or only the 16 CpG sites of Table 7. In other words, no other probes (or no forms or copies of the other entities) for detecting (or measuring) other CpG sites are present in these examples.

The kit may comprise (or further comprise) a label necessary for the detection of the probes (or for the detection of other appropriate entities), for example a selective label as described elsewhere herein. The selective labels may be for example labelled dideoxynucleotides (e.g. ddATP, ddCTP, ddGTP, ddTTP, ddUTP). Such dideoxynucleotides are used in chain extension of the oligonucleotide probe using a polymerase enzyme as described elsewhere herein. The kit may therefore comprise (or further comprise) a polymerase enzyme (e.g. a DNA polymerase enzyme). The kit may comprise (or further comprise) a reagent used in a DNA polymerization process, a DNA hybridization process, and/or a DNA bisulfite conversion process. The kit may comprise (or further comprise) instructions for carrying out the methods of the invention.

Where a probe (or other appropriate entity) is for detecting or measuring the methylation level of a CpG site, it is meant that the probe (or other appropriate entity) is targeted towards said CpG site and not other CpG sites, e.g. is selective for or specific for said CpG site.

The kit may comprise (or further comprise) a means for detecting or measuring (or detecting or measuring the presence of) rheumatoid factor, and/or a means for detecting or measuring (or detecting or measuring the presence of) ACPA (or anti-CCP antibody).

Where the language “for detecting”, “for determining” or “for measuring” or similar is used herein, the terms “suitable for detecting”, “suitable for determining” and “suitable for measuring” or similar are also encompassed.

Appropriate probes for use in the kits of the invention are described elsewhere herein but are conveniently nucleic acid probes. In embodiments the kit contains two different types of probe as described elsewhere herein.

In embodiments, the probes are attached to a solid support or a substrate.

The terms “solid support” and “substrate” as used herein are used interchangeably and refer to a material or group of materials having a rigid or semi-rigid surface or surfaces. In embodiments, at least one surface of the solid support will be substantially flat, although in some embodiments it may be desirable to physically separate synthesis regions for different compounds with, for example, wells, raised regions, pins, etched trenches, or the like. In embodiments, the solid support will take the form of beads, resins, gels, microspheres, or other geometric configurations.

In the kits of the invention, multiple probe types may be included for determining the methylation level of a given CpG site; for example, two probe types may be used, wherein the first probe enables detection of the methylated form of the CpG site (or, depending on the methylation detection protocol used, a derivative of said methylated form of said CpG site) and the second probe enables detection of the unmethylated form of the CpG site (or, depending on the methylation detection protocol used, a derivative of said methylated form of said CpG site, for example a bisulfite-treated or bisulfite-converted form of said CpG site).

In embodiments, the invention provides a panel of CpG sites in accordance with the invention as described elsewhere herein.

In embodiments, the invention provides a panel or set of biomarkers, said panel or set of biomarkers comprising (or consisting of) CpG sites in accordance with the invention as described elsewhere herein.

As used throughout the application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms. Methods comprising certain steps also include, where appropriate, methods consisting of these steps.

Methods of determining the statistical significance of differences between test groups of subjects or differences in levels or values of a particular parameter are well known and documented in the art. For example herein a decrease or increase is generally regarded as statistically significant if a statistical comparison using a significance test such as a Student t-test, Mann-Whitney U Rank-Sum test, chi-square test or Fisher's exact test, one-way ANOVA or two-way ANOVA tests as appropriate, shows a probability value of 50.05.

Further embodiments of the invention are provided in embodiments 1 to 34 below:

1. A method of screening for rheumatoid arthritis in a subject, the method comprising using the methylation levels of at least:
4, 5, 10, 20 or 23 CpG sites selected from the list of CpG site numbers 1 to 145 of Table 9;
   in DNA from a biological sample obtained from the subject in order to screen for rheumatoid arthritis in the subject, wherein said methylation levels are used to provide an indication of the presence or absence of rheumatoid arthritis in the subject.

2. The method of embodiment 1, wherein the at least 4, 5, 10, 20 or 23 CpG sites selected from the list of CpG site numbers 1 to 145 of Table 9 comprise at least:
4, 5, 10, 11, 20, 30 or 31 CpG sites selected from the list of CpG site numbers 1 to 121 of Table 3; and/or
5, 10, 15, 20 or 24 CpG sites selected from the list of CpG site numbers 1 to 24 of Table 5.

3. The method of embodiment 2, wherein the at least 4, 5, 10, 11, 20, 30 or 31 CpG sites selected from the list of CpG site numbers 1 to 121 of Table 3 comprise at least 5, 10, 20, 30 or 31 CpG sites selected from the list of CpG site numbers 1 to 31 of Table 3.

4. The method of embodiment 2 or embodiment 3, wherein the at least 5, 10, 15, 20 or 24 CpG sites selected from the list of CpG site numbers 1 to 24 of Table 5 comprise at least:
5, 10, 15, or 20 CpG sites selected from the list of CpG site numbers 1 to 20 of Table 6; and/or
5, 10, 15, or 16 CpG sites selected from the list of CpG sites numbers 1 to 16 of Table 7.

5. The method of any one of the preceding embodiments, comprising (additionally) using the serology information (preferably the rheumatoid factor status and/or the anti-citrullinated protein antibody status) of the subject in addition to said methylation levels in order to provide said indication.

6. The method of any one of the preceding embodiments, comprising calculating a likelihood of the subject having rheumatoid arthritis as a function of said methylation levels

7. The method of embodiment 6, comprising calculating the likelihood as a function of a linear combination of said methylation levels.

8. The method of embodiment 7, wherein the linear combination of said methylation levels comprises a weighted sum of said methylation levels.

9. The method of any one of embodiments 6 to 8, comprising calculating the likelihood as a logistic function of a linear combination of said methylation levels.

10. The method of any one of embodiments 6 to 9, comprising receiving data representative of said methylation levels, and inputting the data to an algorithm for evaluating said function to determine the likelihood of the subject having rheumatoid arthritis.

11. The method of any one of the preceding embodiments, further comprising making a diagnosis of rheumatoid arthritis based on the methylation levels referred to in any one of the preceding embodiments and/or the likelihood referred to in any one of embodiments 3 to 7, optionally by comparing the methylation levels or likelihood with a cutoff value.

12. The method of any one of the preceding embodiments, wherein said subject is a subject at risk of developing rheumatoid arthritis, or is a subject having or suspected of having rheumatoid arthritis.

13. The method of any one of the preceding embodiments, wherein the biological sample is a blood sample, or a white blood cell sample.

14. The method of any one of the preceding embodiments, further comprising reporting the results of the method, optionally by preparing a written or electronic report.

15. The method of any one of the preceding embodiments, wherein the method is implemented by a computer.

16. The method of any one of the preceding embodiments, further comprising treating said rheumatoid arthritis by therapy or surgery.

17. The method of any one of the preceding embodiments, further comprising altering, ceasing or continuing treatment of said subject.

18. The method of any one of the preceding embodiments, wherein said method comprises a step of measuring the methylation levels before the step of using the methylation levels.

19. A computer program comprising instructions that, when executed by a processing system, cause the processing system to process data representative of methylation levels of at least:
4, 5, 10, 20 or 23 CpG sites selected from the list of CpG site numbers 1 to 145 of Table 9;
   in DNA from a biological sample obtained from a subject, to calculate a likelihood of the subject having rheumatoid arthritis.

20. The computer program of embodiment 19, wherein the at least 4, 5, 10, 20 or 23 CpG sites selected from the list of CpG site numbers 1 to 145 of Table 9 comprise at least:
4, 5, 10, 11, 20, 30 or 31 CpG sites selected from the list of CpG site numbers 1 to 121 of Table 3; and/or
5, 10, 15, 20 or 24 CpG sites selected from the list of CpG site numbers 1 to 24 of Table 5.

21. The computer program of embodiment 20, wherein the at least 4, 5, 10, 11, 20, 30 or 31 CpG sites selected from the list of CpG site numbers 1 to 121 of Table 3 comprise at least 5, 10, 20, 30 or 31 CpG sites selected from the list of CpG site numbers 1 to 31 of Table 3.

22. The computer program of embodiment 20 or embodiment 21, wherein the at least 5, 10, 15, 20 or 24 CpG sites selected from the list of CpG site numbers 1 to 24 of Table 5 comprise at least:
5, 10, 15, or 20 CpG sites selected from the list of CpG site numbers 1 to 20 of Table 6; and/or
5, 10, 15, or 16 CpG sites selected from the list of CpG sites numbers 1 to 16 of Table 7.

23. The computer program of any one of embodiments 20 to 22, comprising instructions that, when executed by a processing system, cause the processing system to process data representative of the rheumatoid factor status and/or the anti-citrullinated protein antibody status of the subject, in addition to the data representative of the methylation levels.

24. The computer program of any one of embodiments 19 to 23, wherein the instructions cause the processing system to calculate the likelihood as a function of said data or methylation levels, preferably as a linear combination of said data or methylation levels.

25. The computer program of embodiment 24, wherein the linear combination of said data or methylation levels comprises a weighted sum of said methylation levels.

26. The computer program of any one of embodiments 19 to 25, wherein the instructions cause the processing system to calculate the likelihood as a logistic function of a linear combination of said data or methylation levels.

27. The computer program of any one of embodiments 24 to 26, wherein the instructions cause the processing system to receive data representative of said data or methylation levels and input the data to an algorithm for evaluating said function to determine the likelihood of the subject having rheumatoid arthritis.

28. A processing system configured to perform the method of any one of embodiments 1 to 18.

29. A method of monitoring rheumatoid arthritis in a subject, the method comprising:
(a) using the methylation levels of at least:
(i) 4, 5, 10, 20 or 23 CpG sites selected from the list of CpG site numbers 1 to 145 of Table 9; or
(ii) 4, 5, 10, 11, 20, 30 or 31 CpG sites selected from the list of CpG site numbers 1 to 121 of Table 3, and/or 5, 10, 15, 20 or 24 CpG sites selected from the list of CpG site numbers 1 to 24 of Table 5; in DNA from a biological sample obtained from the subject at a first time point; and
(b) comparing said methylation levels to the methylation levels of the same at least:
(i) 4, 5, 10, 20 or 23 CpG sites selected from the list of CpG site numbers 1 to 145 of Table 9; or
5, 10, 20, 30 or 31 CpG sites of Table 3, and/or 5, 10, 15, 20 or 24 CpG sites selected from the list of CpG site numbers 1 to 24 of Table 5;
   in DNA from a biological sample obtained from the subject at a second time point.

30. A method of obtaining an indication of the efficacy of a drug which is being used to treat rheumatoid arthritis in a subject, the method comprising:
(a) using the methylation levels of at least:
(i) 4, 5, 10, 20 or 23 CpG sites selected from the list of CpG site numbers 1 to 145 of Table 9; or
(ii) 4, 5, 10, 11, 20, 30 or 31 CpG sites selected from the list of CpG site numbers 1 to 121 of Table 3, and/or 5, 10, 15, 20 or 24 CpG sites selected from the list of CpG site numbers 1 to 24 of Table 5;
in DNA from a biological sample obtained from the subject at a first time point; and
(b) comparing said methylation levels to the methylation levels of the same at least:
(i) 4, 5, 10, 20 or 23 CpG sites selected from the list of CpG site numbers 1 to 145 of Table 9; or
5, 10, 20, 30 or 31 CpG sites of Table 3, and/or 5, 10, 15, 20 or 24 CpG sites of Table 5; within a biological sample obtained from the subject at a second time point, wherein a drug has been administered to the subject in the interval between the first and second time points.

31. The method of embodiment 29 or embodiment 30, wherein a change in the methylation levels between the first and second time points is indicative of a change in severity of rheumatoid arthritis in the subject.

32. A method of treating rheumatoid arthritis in a subject, the method comprising:
(a) obtaining an indication of the presence of rheumatoid arthritis in a subject by performing a method as provided in any one of embodiments 1 to 18; and
(b) administering a treatment appropriate for treating rheumatoid arthritis to the subject if an indication of the presence of rheumatoid arthritis in the subject is obtained, thereby treating rheumatoid arthritis in the subject.

33. A method of screening for rheumatoid arthritis in a subject, the method comprising using the methylation levels of a set of CpG sites in DNA from a biological sample obtained from the subject in order to screen for rheumatoid arthritis in the subject, wherein said methylation levels are indicative of the presence or absence of rheumatoid arthritis in the subject, and wherein said set of CpG sites comprises CpG sites in at least 1 of the following genes: HLA-DQA1, ELANE, HLA-DQA2, HLA-DQB1, CD28 and CD1C.

34. A kit for screening for rheumatoid arthritis in a subject, said kit comprising probes for detecting the methylation levels of at least:
(i) 4, 5, 10, 20 or 23 CpG sites selected from the list of CpG site numbers 1 to 145 of Table 9; or
(ii) 4, 5, 10, 11, 20, 30 or 31 CpG sites selected from the list of CpG site numbers 1 to 121 of Table 3, and/or 5, 10, 15, 20 or 24 CpG sites selected from the list of CpG site numbers 1 to 24 of Table 5;
   in DNA from a biological sample obtained from the subject, optionally wherein the probe (or CpG probe) component of the kit consists of up to 500 different probes or consists of probes for detecting the methylation levels of up to 500 CpG sites.

The invention will now be further described in the following non-limiting Examples with reference to the following figures.

FIG. 1 shows the AUC of an RA classifier of the invention using methylation levels from the 121 CpG sites listed in Table 3. AUC 0.956 (95% confidence intervals of 0.929 to 0.982) on Hold-out set (marked light grey). AUC 0.962 (95% confidence intervals of 0.935 to 0.989) on Dev-set (marked dark grey).

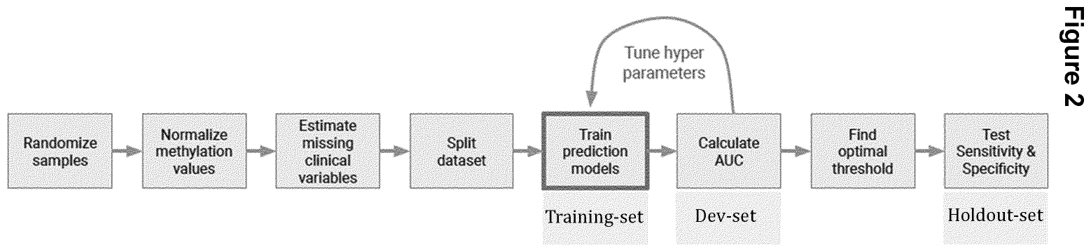

FIG. 2 shows the machine learning pipeline used to identify CpG sites used in the methods of the invention and train models with those CpG sites.

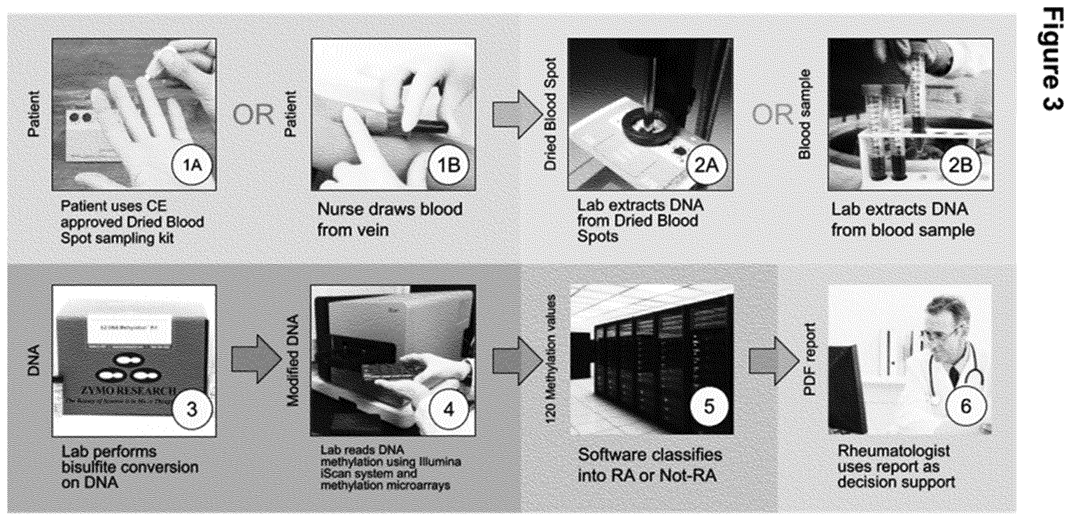

FIG. 3 is a step-by-step flowchart of an exemplary RA diagnostic test from acquisition of the biological sample from the subject to the point of diagnosis by the clinician.

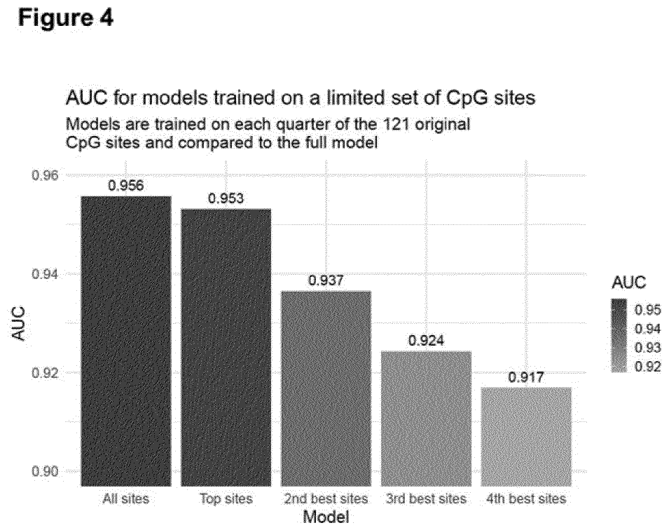

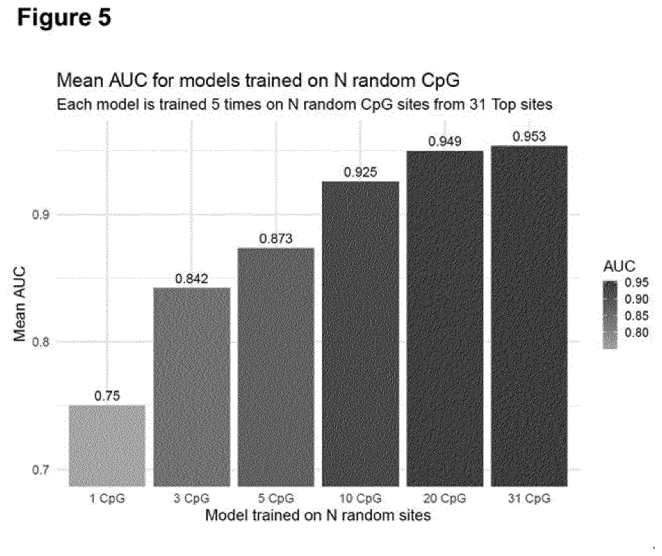

FIGS. 4 and 5 are graphs displaying the AUC values of models trained on a limited subset of CpG sites taken from the list of 121 CpG sites of Table 3.

FIG. 6 shows the AUC of an RA classifier of the invention using methylation levels from the 20 CpG sites listed in Table 6. The standardised coefficients used in the classifier/model in respect of each CpG site are also provided in Table 6, together with the intercept used in the classifier/model. The box embedded in the bottom-right of the graph provides the sensitivity, specificity and balanced accuracy values for the predictor at a cut-off of 0.44 (“Accuracy” in the box means “balanced accuracy”).

FIG. 7 shows the AUC of an RA classifier of the invention using methylation levels from the 16 CpG sites listed in Table 7, plus serology data (RF_pos and CCP_pos) as also listed in Table 7. The standardised coefficients used in the classifier/model in respect of each CpG site and in respect of the serology data are also provided in Table 7, together with the intercept used in the classifier/model. The box embedded in the bottom-right of the graph provides the sensitivity, specificity and balanced accuracy values for the predictor at a cut-off of 0.36 (“Accuracy” in the box means “balanced accuracy”).

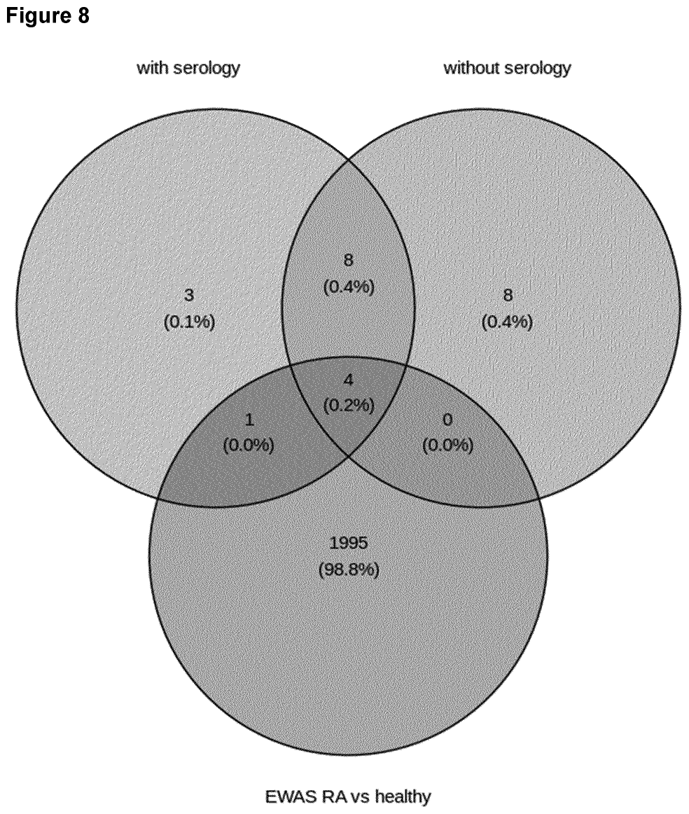

FIG. 8 provides a Venn diagram showing the overlap between the list of CpG sites of Table 6 (“with serology”) and Table 7 (“without serology”), as well as the overlap with the 2000 CpG sites that were identified in the previously conducted EWAS study which had been preferentially weighted in the filtering processes (“EWAS RA vs healthy”; described in Example 8).

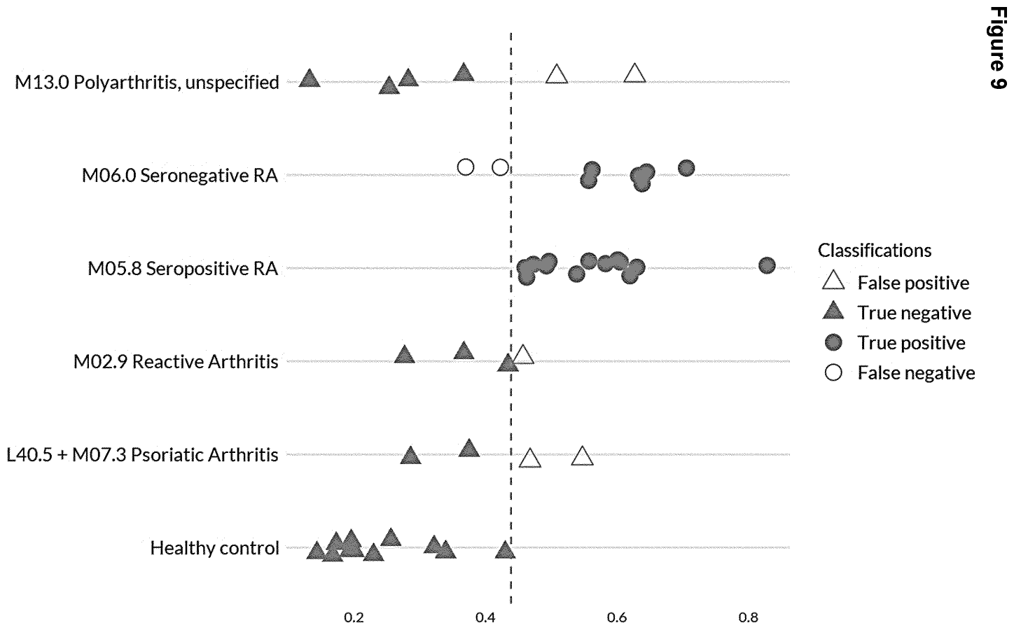

FIG. 9 provides the holdout predictions, by disease group, for the RA predictor using methylation levels from the 20 CpG sites listed in Table 6 (and also using the standardised coefficients and intercept also provided in Table 6). The x-axis is the model's prediction, with a theoretical range of 0-1. The dotted line is the cut-off (0.44).

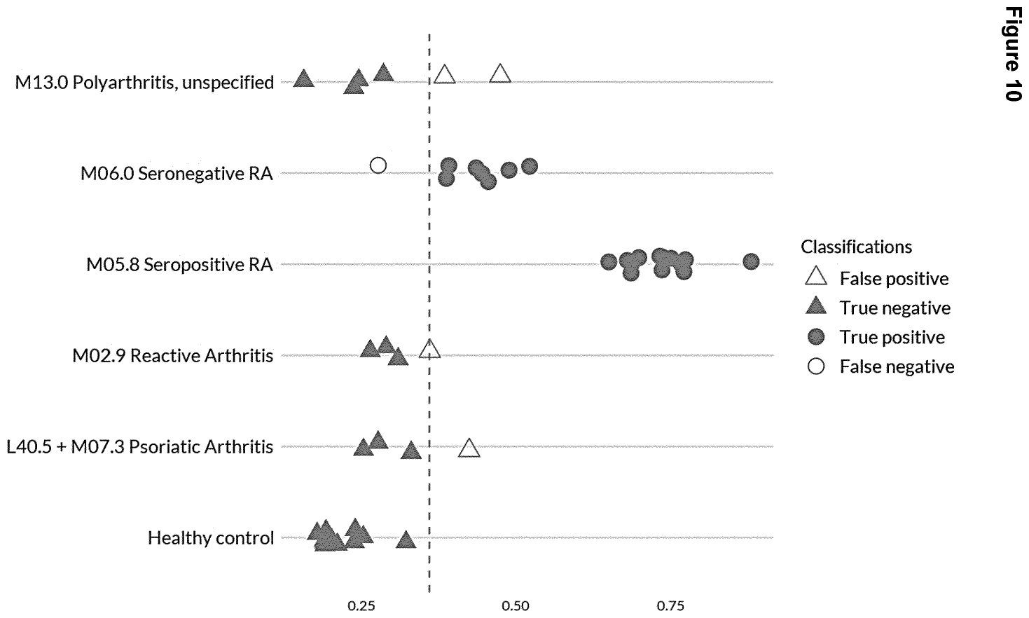

FIG. 10 provides the holdout predictions, by disease group, for the RA predictor using methylation levels from the 16 CpG sites listed in Table 7, plus serology data (RF_pos and CCP_pos) as also listed in Table 7 (and also using the standardised coefficients and intercept also provided in Table 7). The x-axis is the model's prediction, with a theoretical range of 0-1. The dotted line is the cut-off (0.36).

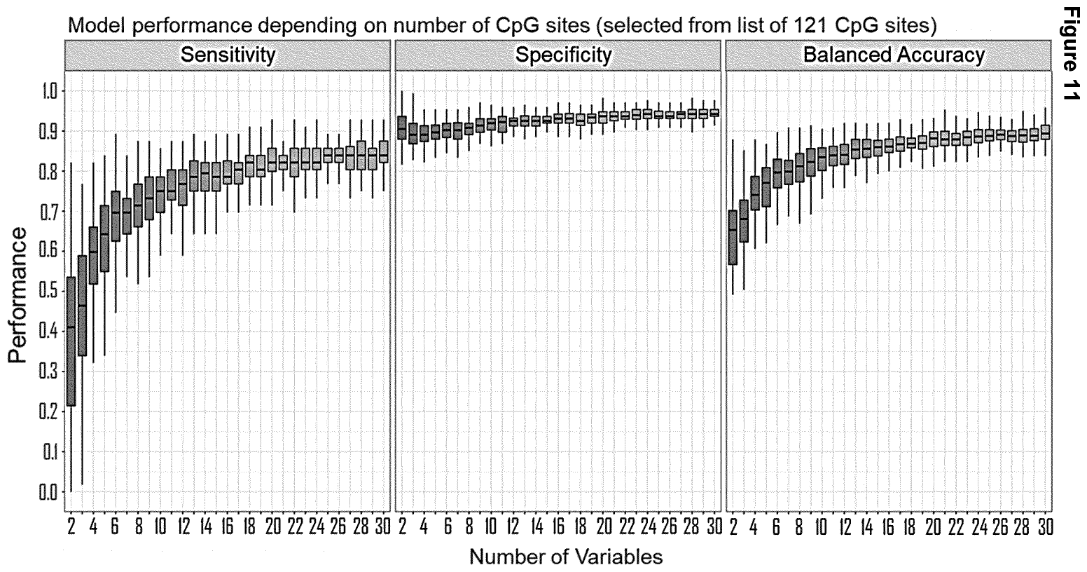

FIG. 11 provides model performance depending on the number of CpG sites used in the model, where the CpG sites are selected from the list of 121 CpG sites of Table 3. Each box-whisker (each bar) shows the results of 100 tested combinations (models). The box is drawn from the first quartile (Q1) to the third quartile (Q3) with a horizontal line drawn in the middle to denote the median. The end of the lower whisker is the minimum performance value of the given number of CpG sites (i.e. the value for the combination of CpG sites (model) which gave the poorest performance), and the end of the upper whisker is the maximum value (i.e. the value for the combination of CpG sites (model) which gave the best performance).

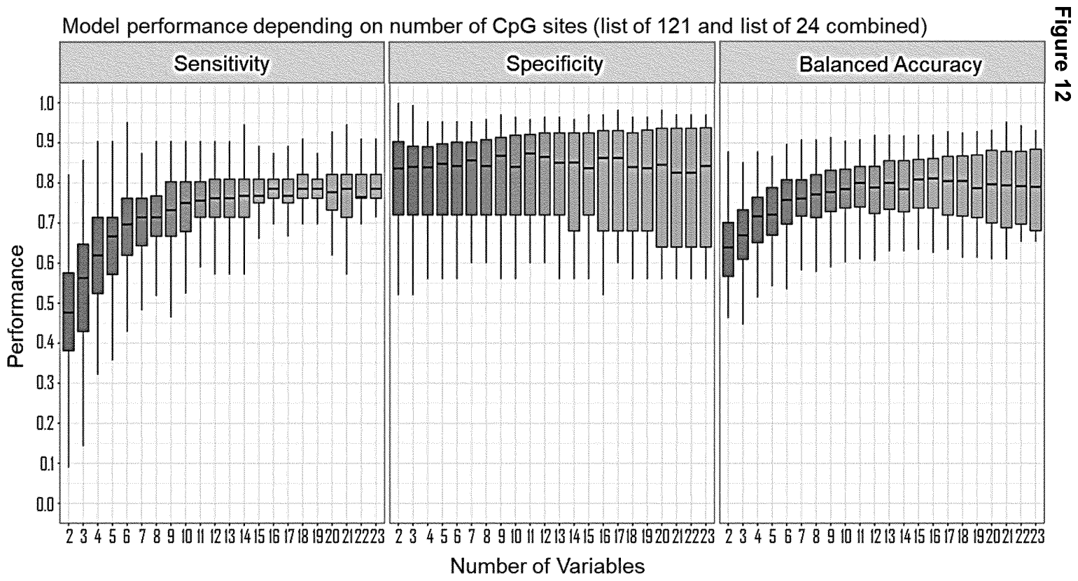

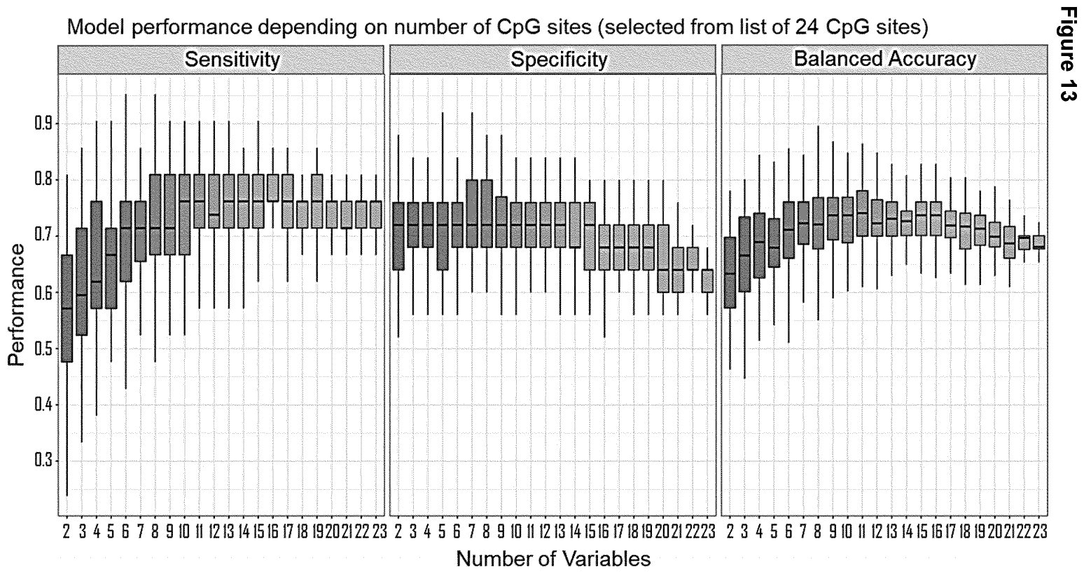

FIG. 12 provides a plot which combines the data in FIGS. 11 and 13 in respect of n=2 to 23. Each box-whisker represents the data in the same manner as described for FIG. 11.

FIG. 13 provides model performance depending on the number of CpG sites used in the model, where the CpG sites are selected from the list of 24 CpG sites of Table 5. Each box-whisker (each bar) shows the results of 100 tested combinations (models) or in the case of n=23, the logical maximum, i.e. 24 models (for n=23 selected from the list of 24 CpG sites). Each box-whisker represents the data in the same manner as described for FIG. 11.

No serology information was used in any of the models presented in FIGS. 11 to 13.

### Example 1—Production of Methylation Level Datasets Used in Examples 2 and 3

The datasets used (shown in Tables 2A and 2B) are publically available and are also described in the literature. In summary, in order to obtain Dataset 1, blood was collected through venepuncture, treated with EDTA and stored at −80° C. until use. For each sample, 1 μg of genomic DNA was bisulfite-converted using an EZ DNA methylation Kit (ZYMO research) according to the manufacturer's recommendations. Converted genomic DNA was eluted in 22 μl of elution buffer. DNA methylation level was measured using the Illumina Infinium HD Methylation Assay (Illumina) according to the manufacturer's instructions. Briefly, 4 μl of bisulfite-converted DNA was isothermally amplified overnight (20-24 hours) and fragmented enzymatically. Precipitated DNA was resuspended in hybridization buffer and dispensed onto the Infinium HumanMethylation450 BeadChips (12 samples/chip) using a Freedom EVO robot (Tecan). The hybridization procedure was performed at 48° C. overnight (16-20 hours) using an Illumina Hybridization oven. After hybridization, free DNA was washed away and the BeadChips were processed through a single nucleotide extension followed by immunohistochemistry staining using a Freedom EVO robot (Tecan). Finally, the BeadChips were imaged using an Illumina iScan.

Methods of obtaining the other 4 datasets followed essentially the same protocol as above.

All 5 datasets therefore are derived from measurement of methylation levels in DNA from white blood cells from blood samples.

### Example 2—Method of Identifying the 121 CpG Sites of Table 3, and Method of Training and Testing Models Using the 121 CpG Sites or Subsets Thereof

Machine learning techniques were used to identify the 121 CpG sites of Table 3. The machine learning techniques used the five datasets described in Tables 2A and 2B, which comprise not only methylation levels of CpG sites from subjects having rheumatoid arthritis and healthy subjects, but also methylation levels of CpG sites from subjects not having rheumatoid arthritis but having a disease similar to rheumatoid arthritis. As set out in FIG. 2, this process comprised randomizing the samples in the datasets, normalising the methylation values, estimating missing clinical variables, splitting the dataset(s) into a training dataset, a development dataset and a hold-out dataset; training the prediction model and tuning the hyperparameters using the training set and development set; selecting a cutoff value; and testing the algorithm with the hold-out dataset. The holdout set was used solely for the final testing and was not exposed to the model beforehand.

The inventors of the present invention identified, out of 450,000 CpG sites, a combination of 121 CpG sites in particular whose methylation levels were most indicative of the presence or absence of rheumatoid arthritis. These 121 CpG sites are recited in Table 3. This combination of 121 CpG sites of Table 3 yielded the highest AUC value of all the combinations of CpG sites tested (however, excellent AUC values were still achieved (greater than 0.9) when reduced subsets of the 121 CpG sites of Table 3 were used, e.g. 10 or 20 CpG sites, even down to 3 or 5 CpG sites—see FIGS. 4 and 5).

### Example 3—Epigenetic Test for Rheumatoid Arthritis Using the 121 CpG Sites of Table 3

The inventors have developed a test for diagnosing Rheumatoid Arthritis (RA) by reading the DNA methylation level in white blood cells at a specific set of 121 CpG sites (listed in Table 3), and combining these values into a score using a mathematical formula that classifies the test sample into either RA-positive or RA-negative.

The mathematical formula is in the form of a multiple logistic regression such that

$$p = \frac{1}{1 + {b}^{- ( {β}_{0} + {β}_{1} {x}_{1} + {β}_{2} {x}_{2} ++ {β}_{m} {x}_{m} )}}$$

where
p is the probability of RA-positive (a value between 0 and 1)
b is e
β₀ is the regression intercept
β₁ is the weight for CpG-site 1
x₁ is the methylation level of CpG-site 1
βm is the weight for CpG-site m
xₘ is the methylation level of CpG-site m

When tested on a Hold-out set (See Table 1) using a cutoff of p>0.5 for RA-positive, the test has an overall accuracy of 0.87, a sensitivity of 0.76, and a specificity of 0.92. The AUC on the Hold-out set was 0.956 (see FIG. 1).

TABLE 1
Sensitivity and specificity on Hold-out set (N = 173)

| Test category - RA | Total N | Positive N | Sensitivity |
|---|---|---|---|
| Rheumatoid Arthritis | 55 | 42 | 0.76 |

| Test category - Non-RA | Total N | Positive N | Specificity |
|---|---|---|---|
| Mix - see below | 118 | 10 | 0.92 |
| Crohn's Disease | 14 | 0 | 1.00 |
| Ulcerative Colitis | 23 | 0 | 1.00 |
| Multiple Sclerosis | 2 | 0 | 1.00 |
| Pulmonary Tuberculosis | 1 | 0 | 1.00 |
| Sepsis | 4 | 0 | 1.00 |
| Healthy Symptomatic IBD | 11 | 0 | 1.00 |
| Healthy Control | 63 | 10 | 0.84 |

FIG. 1 shows the AUC of an RA classifier of the invention. AUC 0.956 (95% confidence intervals of 0.929 to 0.982) on Hold-out set (marked light grey). AUC 0.962 (95% confidence intervals of 0.935 to 0.989) on Dev-set (marked dark grey).

The 121 CpG sites selected for this test (the CpG sites listed in Table 3), and the weights for each of these sites (“Standardized Coefficients” column of Table 3), were found using machine learning techniques that analyzed and trained on 5 case-control methylation datasets (See Table 2A and 2B) as described in Example 2. The methylation datasets included healthy and symptomatic controls, RA cases, and cases for other diseases known to cause similar immune response to that of RA. Using this mix of methylation datasets increases the test's robustness (less false positives and false negatives) and increases the likelihood of CpG sites associated solely with the RA diseases being selected.

The model was trained on 5 datasets in total (Tables 2A and 2B) using machine learning. Each dataset contains, for each individual (human), 450,000 methylation levels and a diagnosis (RA or not, MS or not, etc.). The dataset was split into three parts consisting of a Training-set, a Dev-set, and a Hold-out set. The Training-set was used for training of the algorithm, the Dev-set was used to find the optimal hyperparameters, and the hold-out set was used solely for the final testing.

The machine learning technique was used to produce a standardized logistic regression coefficient (i.e. normalised weight) for each CpG site. Each of the 121 CpG sites and its standardized logistic regression coefficient (or weight) is provided in Table 3 (referred to in Table 3 simply as “Standardized Coefficients”). The name of each CpG site is provided in the “feature” column. For ease of reference herein, each CpG site has been given a number from 1 to 121 for identification purposes, as provided in the “CpG site number” column. This list also shows which gene the CpG site is associated with, and which chromosome the CpG site is situated on. The list is sorted from most important (CpG site number 1) to least important (CpG site number 121), in order of the size of the standardized logistic regression coefficient for each CpG site. The last row of the list of Table 3 provides the regression intercept.

The same technique as described above has been followed for the model using the list of 20 (Table 6), the model using the list of 16 (Table 7) and the model using the list of 24 (Table 5).

TABLE 2A
Datasets used for training, testing and hold-out (N = 1154)

| | | | | | | | Healthy | |
|---|---|---|---|---|---|---|---|---|
| | Rheumatoid | Crohn's | Ulcerative | Multiple | Pulmonary | | Symptomatic | Healthy |
| Dataset | Arthritis | Disease | Colitis | Sclerosis | Tuberculosis | Sepsis | IBD | Control |
| Dataset 1 | ✓ | | | | | | | ✓ |
| Dataset 2 | | ✓ | ✓ | | | | ✓ | ✓ |
| Dataset 3 | | | | ✓ | | | | |
| Dataset 4 | | | | | ✓ | | | ✓ |
| Dataset 5 | | | | | | ✓ | | ✓ |
| N | 354 | 103 | 101 | 52 | 3 | 17 | 75 | 449 |

TABLE 2B

| | Tissue | | Methylation | |
|---|---|---|---|---|
| Dataset | type | Samples | array type | Age |
| Dataset 1 | Whole blood | 689 | 450k | 18 to 70 |
| | | | | years old |
| Dataset 2 | Whole blood | 384 | 450k | 17 to 79 |
| | | | | years old |
| Dataset 3 | Whole blood, | 95 | 450k | 26 to 59 |
| | Monocytes | | | years old |
| Dataset 4 | Whole blood | 6 | EPIC | Not found |
| Dataset 5 | Whole blood | 23 | EPIC | 5 to 7 |
| | | | | days old |

Table 4 provides a comparison of the performance of present method with methods of the prior art.

TABLE 4
Our test compared to RF and ACPA

| Lab test name | Sensitivity | Specificity |
|---|---|---|
| RF (Rheumatoid factor) | 68.00% | 85.00% |
| ACPA | 68.00% | 95.00% |
| Age Labs (balanced | 76.00% | 92.00% |
| threshold) | | |
| Age Labs (optimized | 58.00% | 97.00% |
| for specificity) | | |

FIG. 3 shows an exemplary RA test step-by-step. The test is performed in four parts (See FIG. 3)
Blood is drawn from the patient (parts 1A and 1B)
DNA from white blood cells is extracted (parts 2A and 2B)
Methylation levels at 121 CpG sites of Table 3 are measured (parts 3 and 4)
Algorithm classifies into RA-positive or RA-negative (parts 5 and 6) using the methylation levels from 121 specific CpG sites of Table 3 in combination with their weights.

### Example 4—Testing Performance of Models Trained on Limited Sets of CpG Sites

In order to test the number of CpG sites that could be relied upon to produce a high quality model, four models were trained separately on each quarter of the 121 original CpG sites of Table 3 (top 1-31, 32-61, 62-91, 92-121). The method of training was otherwise the same as described in Examples 2 and 3. It was demonstrated that each of the four models exhibited an AUC value above 0.9, i.e. “outstanding” quality (FIG. 4).

### Discussion

This demonstrates that a high quality model/algorithm of the invention can be generated which trains on and uses methylation levels of only 31 or 30 of the list of 121 CpG sites of Table 3. Given that an AUC value of 0.7 or greater for a diagnostic test is generally considered to indicate “acceptable” quality, it will be understood that the methylation levels of significantly fewer than 31 or 30 CpG sites selected from the list of 121 CpG sites of Table 3 could be used to achieve a workable model of the present invention.

### Example 5—Testing Performance of Models Trained on Combinations of CpG Site Numbers 1-31 of Table 3

In order to test the smallest numbers of CpG sites that could be relied upon to produce a high quality model, models were trained separately using various combinations of CpG site numbers 1 to 31 of Table 3. The method of training was otherwise the same as described in Examples 2 and 3. The average AUC value for each number of CpG sites used is displayed in FIG. 5.

### Discussion

This demonstrates that a high quality model/algorithm of the invention can be generated which trains on and uses methylation levels of combinations of only 3, 4, 5, 10, 11 or 20 of CpG site numbers 1 to 31 of Table 3. An AUC value of 0.75 is obtained even when only one site is used. Given that an AUC value of 0.7 or greater for a diagnostic test is generally considered to indicate “acceptable” quality, it will be understood that the methylation levels of significantly fewer than 31 CpG sites selected from the list of 121 CpG sites of Table 3, e.g. as low as 3, 5, 10 or 20, could be used to achieve a highly workable model of the present invention.

### Example 6—Identification of Genes and Pathways Involved in the RA Predictor

KEGG and GO pathway analysis was performed on the 121 CpG sites identified in Example 2 (i.e. the CpG sites listed in Table 3). The function gometh from missMethyl was used. missMethyl is a library for the analysis of Illumina's 450K human methylation BeadChip. The CpG sites were mapped to Entrez Gene IDs, and tests for GO term or KEGG pathway enrichment were performed using a hypergeometric test, taking into account the number of CpG sites per gene on the 450K array. This analysis provides an understanding of which genes and pathways the RA predictor of the present invention involves.

KEGG is a database resource for understanding high-level functions and utilities of the biological system, such as the cell, the organism and the ecosystem, from molecular-level information, especially large-scale molecular datasets generated by genome sequencing and other high-throughput experimental technologies.

The Gene Ontology (GO) knowledgebase is the world's largest source of information on the functions of genes. This knowledge is both human-readable and machine-readable, and is a foundation for computational analysis of large-scale molecular biology and genetics experiments in biomedical research.

With a false discovery rate of 5% the inventors found 10 KEGG pathways and 0 GO pathways involved.

The genes associated with the CpG sites included in the predictor are enriched in biological pathways related to RA and other immunological diseases indicating that aberrant methylation of regulatory regions associated with these genes are important for the functioning of the predictor.

In the 10 pathways found by the KEGG analysis, the following six genes were found to be differentially methylated: HLA-DQA1, ELANE, HLA-DQA2, HLA-DQB1, CD28 and CD1C. CpG sites found within these genes may thus be useful in the diagnosis of rheumatoid arthritis as explained elsewhere herein.

This section lists all the genes found from CpG site numbers 1 to 10 of Table 3, together with a brief explanation of the function of the gene and how it might be related to RA.

Gene: NLRC5 (cg16411857)

This gene plays a role in antiviral immunology through the inhibition of NF-κB. Upregulation of this gene has potential for treatment of rheumatoid arthritis.

Gene: SMARCA4 (cg22898082)

This gene encodes an ATPase that is involved in ATP dependent chromatin modelling and is important for transcriptional activation of normally repressed genes. The gene is associated with psoriasis arthritis, and is involved in TGFbeta and interferon pathways. It is suggested that an epigenetic imbalance of chromatin remodelling factors involved in inflammation pathways may have a potential role in PsA/psoriasis immunopathogenesis (Vecellio M et al., Annals of the Rheumatic Diseases 2021; 80:410-411). The findings of the present invention suggest that this gene or such dysregulation also appears to be important in rheumatoid arthritis.

Gene: HLA-DQA2 (cg05428452)

This HLA type is associated with RA.

Gene: SAFB/SAFB2 (cg04708340)

These genes are involved in chromatin or nuclear scaffolding. They are associated with the IL1 pathway, a key mediator of RA. Downregulation of these genes activates the NF-κB pathway (which is associated with stress response). Incorrect regulation of NF-κB has been linked to cancer, inflammatory and autoimmune disease. Anti NF-κB therapy has been suggested as treatment for RA, and the pathway is important for this disease.

Gene: SMU1 (cg13714271)

A gene involved in the creation of messenger RNA. The gene is required for normal cell division.

Gene: BCAS4 (cg14950044)

Breast carcinoma amplified sequence 4 is a gene that is expressed in blood tissue and other tissues. Its function is not fully known. It is overexpressed in breast cancer and other diseases and may have a role in disease. The findings of the present invention suggest that this gene also appears to be important in rheumatoid arthritis.

Gene: TH (cg19878200)

The Tyrosine Hydroxylase gene codes for a protein converting tyrosine to dopamine and is the rate-limiting enzyme in the synthesis of catecholamines (i.e adrenaline-like molecules associated with stress). The sympathetic nervous system is involved in joint inflammation. TH positive cells in the synovial (joint) tissue have been shown to be antinflammatory in experimental arthritis. The findings of the present invention suggest that this gene also appears to be important in rheumatoid arthritis.

Gene: KIF16B (cg10003549)

The Kinesin-Like Protein is a protein involved in intracellular trafficking.

This gene has been found to be differentially methylated in eroded vs intact cartilage in osteoarthritis. The findings of the present invention suggest that this gene also appears to be important in rheumatoid arthritis.

CpG Site: cg00966255

This CpG site has been identified as possibly associated with psoriasis arthritis, a degenerative joint disease and a differential diagnosis to RA. It is also listed as a B-cell specific CpG site (B-cells produce antibodies and are a part of the immune system). The findings of the present invention suggest that this site/gene associated with this site also appears to be important in rheumatoid arthritis.

CpG Site: cg11164639 Identified as associated with age in human saliva. The findings of the present invention suggest that this site/gene associated with this site also appears to be important in rheumatoid arthritis.

This section lists the six genes identified from the KEGG analysis of Example 6.

### Gene: HLA-DQA1

This gene is associated with susceptibility to RA. The data of the present invention suggests that aberrant methylation of this gene may also be associated with RA.

### Gene: ELANE

This gene encodes neutrophil elastase, an enzyme secreted by neutrophils, an important and abundant cell type of the immune system. The gene has been associated with response to treatment in RA patients previously and the data of the present invention suggests that aberrant methylation of this gene may also be associated with RA.

### Gene: HLA-DQA2

This HLA type is associated with RA. The data of the present invention suggests that aberrant methylation of this gene may also be associated with RA.

### Gene: HLA-DQB1

Polymorphisms in this gene have been associated (both positively and negatively) with RA. The data of the present invention suggests that aberrant methylation of this gene may also be associated with RA.

### Gene: CD28

The Cluster of Differentiation gene 28 is a protein expressed on the surface of T-cells involved in regulating the immune system. Stimulation of T-cells through this receptor can provide a potent signal for the production of inflammatory cytokines which of course are involved in RA. The data of the present invention suggests that aberrant methylation of this gene may also be associated with RA.

### Gene: CD1C

Pro T cells (DN4 type) CD1c⁺ myeloid dendritic cells in synovial fluid are involved in the inflammatory cascade intra-articularly by the secretion of specific T cell-attracting chemokines and the activation of self-reactive T cells. The synovial fluid is the fluid in the joints and is involved in joint diseases like RA. The data of the present invention suggests that aberrant methylation of this gene may also be associated with RA.

### Example 7—Production of Methylation Level Datasets Used in Examples 8 and Above

The datasets were generated using the Illumina Infinium MethylationEPIC Kit Array, which covers 850 k CpG sites. The datasets consisted of a cohort of 94 RA patients (58 seropositive for RA, 36 seronegative for RA), combined with 74 patients suffering from other arthritic diseases and 50 healthy controls. Methods of obtaining the datasets used in Examples 8 and above followed essentially the same protocol as provided in Example 1. The datasets therefore were derived from measurement of methylation levels in DNA from blood samples (i.e. from white blood cells).

### Example 8—Method of Identifying the 24 CpG Sites of Table 5, and Method of Training and Testing Models Using the 24 CpG Sites or Subsets Thereof

Machine learning techniques were used to identify the 24 CpG sites of Table 5. The machine learning techniques used the datasets described in Example 7, which comprise not only methylation levels of CpG sites from subjects having seropositive rheumatoid arthritis and healthy subjects, but also methylation levels of CpG sites from seronegative RA subjects and subjects not having rheumatoid arthritis but having different arthritic diseases.

The process for identification of the 24 CpG sites was as set out in FIG. 2 and as described in Example 2. Thus, importantly, the holdout set was used solely for the final testing and was not exposed to the model beforehand.

The 24 CpG sites were identified from the 850 k CpG sites on the EPIC array using the datasets described above. This was achieved through the training of two models. One of the model training processes utilized serology information by adding rheumatoid factor (RF) and anti-cyclic citrullinated peptide (anti-CCP) test results as additional predictor variables to the retained CpG sites. The other filtering process (model training process) made no use of these two additional variables, because they may not be available for every patient in practice. Both models were trained to predict RA diagnosis and were restricted to a maximum of 25 CpG sites.

Both training processes involved preferentially weighting 2000 CpG sites which had been found—in an EWAS experiment using Dataset 1 (described in Tables 2A and 2B)—to be most significantly differentially methylated between RA patients and healthy controls.

The model using serology information included 16 CpG sites as predictor variables (Table 7); the model that did not use serology information (i.e. used only CpG sites) included 20 CpG sites (Table 6). 12 of these sites overlapped between the two models for a total of 24 sites used (Table 5). 5 of these 24 sites (cg04399899, cg07329251, cg07930752, cg10266904, cg27552857) were also found in the aforementioned EWAS analysis in the 450 k data, demonstrating reproducibility across datasets and arrays (see FIG. 8).

The models combine the values into a score using a mathematical formula that classifies the test sample into either RA-positive or RA-negative. The mathematical formula is in the form of a multiple logistic regression as described in Example 3.

The model using the combination of 20 CpG sites (Table 6) yielded an AUC value of 0.91 (outstanding performance), while the model using the combination of 16 CpG sites with serology information (Table 7) yielded an AUC value of 0.97 (outstanding performance). Further performance metrics and data for the two models are provided in Table 8 (“serology used” cut-off=0.36; “serology not used” cut-off=0.44), and FIGS. 6, 7, 9 and 10 help to visualise these data.

TABLE 8

| | Serology Used | Serology Not Used |
|---|---|---|
| Model | (list of 16) | (list of 20) |
| AUC | 0.97 | 0.91 |
| Sensitivity | 0.95 | 0.86 |
| Specificity | 0.84 | 0.8 |
| Balanced Accuracy | 0.89 | 0.83 |
| Accuracy Seronegative | 0.88 | 0.75 |
| Accuracy Seropositive | 1 | 0.92 |
| Accuracy Healthy | 1 | 1 |
| Accuracy Other Arthritis | 0.71 | 0.64 |

A model using all 24 sites together (without serology information) was also generated (Table 5), yielding a sensitivity of 0.81, a specificity of 0.68, and a balanced accuracy of 0.75 (excellent performance).

From these data it is demonstrated that these 24 CpG sites may be used to predict rheumatoid arthritis, and are especially suited to identify seronegative RA patients and also distinguish RA from other arthritic diseases.

### Example 9—Additional Testing of Performance of Models Trained on Limited Sets of CpG Sites

Models were trained with different numbers of CpG sites randomly selected from the list of 121 sites of Table 3 or the list of 24 sites of Table 5. For each number of CpG sites, a total of 100 models were trained (or the logical maximum, e.g. 24 models for n=23 selected from the list of 24 CpG sites), after which the sensitivity, specificity, and balanced accuracy for each model was determined. A box-whisker plot for each number of sites (n) is provided in FIG. 11 for the list of 121 sites (n=2 to 30), and FIG. 13 for the list of 24 (n=2 to 23). FIG. 12 provides a plot which combines the data in FIGS. 11 and 13 in respect of n=2 to 23. No serology information was used in any of the models represented in FIGS. 11 to 13.

The data demonstrates that even with a small number of sites, a balanced accuracy of 0.6 or higher can be achieved (indicating a working predictor) or even a balanced accuracy of 0.75 or higher (indicating excellent performance) using combinations drawn from the 145 CpG sites (Table 9) identified above.

### LIST OF RELEVANT TECHNICAL TERMS

Rheumatoid arthritis (RA)—a long-term autoimmune disorder that primarily affects joints
Seronegative—a person with RA where both Rheumatoid Factor (RF) and ACPA comes out negative
Seropositive—a person with RA where either RF or ACPA comes out positive
Epigenetics—the study of changes in organisms caused by modification of gene expression rather than alteration of the DNA itself
DNA methylation—a type of epigenetic modification where a methyl group is added to DNA
CpG site—a position in the DNA where a cytosine nucleotide (C) is followed by a guanine nucleotide (G) along the sequence of bases
Methylation level—a measure of how much a CpG site is methylated in a set of cells
Weight—the regression coefficient that the methylation level for a CpG site is multiplied by
Multiple regression—a statistical technique that uses several explanatory variables to predict the outcome of a response variable
Logistic regression—a statistical model that in its basic form uses a logistic function to model a binary dependent variable
Machine learning (ML)—the study of computer algorithms that improve automatically through experience
Algorithm—a machine learning algorithm is not explicitly programmed, but built from sample data, aka Training-set, in order to make predictions
Hyperparameters—parameters that are set before the machine learning process begins, they are tunable and can directly affect how well a model trains
Prediction model—see Algorithm
Elastic net—is a regression method that combines the L1 and L2 penalties of the lasso and ridge regression methods
AUC—stands for “Area under the ROC Curve” and provides an aggregate measure of performance across all possible classification thresholds
Sensitivity—the ability of a test to correctly identify those with the disease (true positive rate)
Specificity—the ability of the test to correctly identify those without the disease (true negative rate)
Overall accuracy—the proportion of correct predictions (both true positives and true negatives) among the total number of cases examined

### TABLES

Tables of CpG sites referenced in the Examples and in the main part of the description are provided below. In these Tables, the CpG ID number (e.g. cg20843080) of each CpG site is provided in the “feature” column. The chromosome on which each of the 145 CpG sites is located is provided in the “chr” column of Table 9, and the position of the CpG site on the chromosome is provided in the “pos” column of Table 9. For the purposes of referring to each CpG site in a concise manner in the present specification, each CpG site in each Table has been assigned a “CpG site number”. For example, CpG site “cg20843080” can alternatively be referred to as “CpG site number 1 of Table 5”, or as “CpG site number 122 of Table 9”, and so on.

TABLE 3
list of 121 CpG sites, ranked by standardised
coefficient, and intercept

| CpG site | | | | Standardized |
|---|---|---|---|---|
| number | feature | coefficient | SD | Coefficients |
| 1 | cg16411857 | −4.1756464 | 0.0784618 | −0.3276286 |
| 2 | cg22898082 | 4.1061323 | 0.0750094 | 0.3079986 |
| 3 | cg05428452 | −2.1815531 | 0.1380286 | −0.3011166 |
| 4 | cg04708340 | 9.7791570 | 0.0270290 | 0.2643204 |
| 5 | cg13714271 | −3.5481544 | 0.0435502 | −0.1545228 |
| 6 | cg14950044 | −2.7109935 | 0.0510894 | −0.1385030 |
| 7 | cg19878200 | 2.3775050 | 0.0534130 | 0.1269897 |
| 8 | cg10003549 | 2.4760022 | 0.0500878 | 0.1240175 |
| 9 | cg00966255 | 3.0331106 | 0.0400154 | 0.1213712 |
| 10 | cg11164639 | −0.9544200 | 0.1227148 | −0.1171214 |
| 11 | cg26164488 | −1.0471769 | 0.1094575 | −0.1146214 |
| 12 | cg24514600 | −0.7757710 | 0.1414582 | −0.1097392 |
| 13 | cg27008565 | 1.6870669 | 0.0614154 | 0.1036118 |
| 14 | cg27095222 | −1.3072791 | 0.0781274 | −0.1021344 |
| 15 | cg02030958 | 1.2407739 | 0.0787531 | 0.0977147 |
| 16 | cg13651908 | 1.1582076 | 0.0800500 | 0.0927145 |
| 17 | cg21486694 | −1.3277603 | 0.0697881 | −0.0926619 |
| 18 | cg09873215 | −1.1545295 | 0.0795684 | −0.0918640 |
| 19 | cg25560009 | 1.5189593 | 0.0564716 | 0.0857781 |
| 20 | cg04917446 | −0.7799501 | 0.1089114 | −0.0849454 |
| 21 | cg21889472 | 3.5879597 | 0.0235364 | 0.0844478 |
| 22 | cg13081526 | −0.3552731 | 0.2360031 | −0.0838456 |
| 23 | cg19945060 | −1.5210356 | 0.0544990 | −0.0828949 |
| 24 | cg08082299 | 3.0302415 | 0.0257952 | 0.0781657 |
| 25 | cg14026109 | 2.6288812 | 0.0276220 | 0.0726149 |
| 26 | cg06387622 | −1.4535126 | 0.0487094 | −0.0707998 |
| 27 | cg00119117 | 3.2716171 | 0.0214938 | 0.0703193 |
| 28 | cg03441250 | 1.2918621 | 0.0512953 | 0.0662664 |
| 29 | cg19544767 | −0.4246759 | 0.1556843 | −0.0661154 |
| 30 | cg09455126 | −1.5775709 | 0.0412019 | −0.0649989 |
| 31 | cg07231053 | 2.2420478 | 0.0281912 | 0.0632061 |
| 32 | cg01534423 | 0.7361383 | 0.0850526 | 0.0626105 |
| 33 | cg23985214 | 2.8235188 | 0.0219881 | 0.0620839 |
| 34 | cg08447792 | 1.5250962 | 0.0404206 | 0.0616453 |
| 35 | cg11646192 | −1.8801058 | 0.0318139 | −0.0598135 |
| 36 | cg14052235 | −1.0531824 | 0.0555258 | −0.0584788 |
| 37 | cg18036763 | 0.4144726 | 0.1405868 | 0.0582694 |
| 38 | cg26043955 | 0.9533226 | 0.0606983 | 0.0578651 |
| 39 | cg20726993 | 0.5489481 | 0.1010769 | 0.0554860 |
| 40 | cg06544989 | 1.4625033 | 0.0354745 | 0.0518816 |
| 41 | cg12359114 | 4.5328257 | 0.0114218 | 0.0517732 |
| 42 | cg05370853 | −0.4028788 | 0.1268896 | −0.0511211 |
| 43 | cg12192813 | 0.3719769 | 0.1361588 | 0.0506479 |
| 44 | cg22424532 | 0.8537212 | 0.0584313 | 0.0498841 |
| 45 | cg02539793 | −1.0510655 | 0.0461525 | −0.0485093 |
| 46 | cg00672574 | 2.6122919 | 0.0185571 | 0.0484766 |
| 47 | cg18734095 | 0.7108151 | 0.0650349 | 0.0462278 |
| 48 | cg01120173 | −0.5305167 | 0.0825299 | −0.0437835 |
| 49 | cg14480789 | −0.8935166 | 0.0442446 | −0.0395333 |
| 50 | cg21106695 | −0.6405581 | 0.0615176 | −0.0394056 |
| 51 | cg01849466 | 1.4908122 | 0.0255432 | 0.0380802 |
| 52 | cg24931954 | 1.3849647 | 0.0269482 | 0.0373223 |
| 53 | cg15674266 | 2.3574789 | 0.0153619 | 0.0362154 |
| 54 | cg05225373 | 1.8600370 | 0.0191112 | 0.0355475 |
| 55 | cg16078521 | 0.3246279 | 0.1072695 | 0.0348227 |
| 56 | cg15996459 | −0.3505391 | 0.0983439 | −0.0344734 |
| 57 | cg25666547 | −0.4337908 | 0.0737423 | −0.0319887 |
| 58 | cg20000464 | −0.3542605 | 0.0895261 | −0.0317155 |
| 59 | cg06118351 | 0.1449376 | 0.2143214 | 0.0310632 |
| 60 | cg00249093 | −0.4621585 | 0.0669488 | −0.0309410 |
| 61 | cg22125253 | −0.9471381 | 0.0324836 | −0.0307665 |
| 62 | cg14883135 | −0.1402116 | 0.2193837 | −0.0307601 |
| 63 | cg19057882 | 0.8952631 | 0.0339222 | 0.0303693 |
| 64 | cg17131070 | 1.3050135 | 0.0230711 | 0.0301081 |
| 65 | cg19273683 | 0.4679458 | 0.0617985 | 0.0289183 |
| 66 | cg08707123 | 1.0304354 | 0.0273880 | 0.0282216 |
| 67 | cg19204693 | −1.2248822 | 0.0230352 | −0.0282154 |
| 68 | cg13862851 | −0.3867594 | 0.0724760 | −0.0280308 |
| 69 | cg11628021 | −0.9153413 | 0.0304920 | −0.0279106 |
| 70 | cg11925488 | −0.5976455 | 0.0459218 | −0.0274449 |
| 71 | cg05522848 | −0.5239664 | 0.0508095 | −0.0266225 |
| 72 | cg24702253 | 0.1498768 | 0.1727270 | 0.0258878 |
| 73 | cg17903019 | 0.3233715 | 0.0794562 | 0.0256939 |
| 74 | cg21853806 | 0.8313267 | 0.0286624 | 0.0238278 |
| 75 | cg21247338 | 0.9981259 | 0.0233773 | 0.0233335 |
| 76 | cg05494709 | −0.4049457 | 0.0571718 | −0.0231515 |
| 77 | cg26720338 | 0.8513407 | 0.0254268 | 0.0216468 |
| 78 | cg09138892 | −0.2647757 | 0.0812260 | −0.0215067 |
| 79 | cg14882966 | −0.2606157 | 0.0823249 | −0.0214552 |
| 80 | cg26561212 | −0.4065772 | 0.0526750 | −0.0214165 |
| 81 | cg26019250 | 0.3866498 | 0.0535599 | 0.0207089 |
| 82 | cg06311103 | −0.7037822 | 0.0288448 | −0.0203005 |
| 83 | cg01437439 | 0.7223991 | 0.0279329 | 0.0201787 |
| 84 | cg10905401 | −0.5670471 | 0.0346621 | −0.0196550 |
| 85 | cg05322982 | −0.8351009 | 0.0218983 | −0.0182873 |
| 86 | cg01558909 | 0.5071940 | 0.0324347 | 0.0164507 |
| 87 | cg23574427 | −0.1030936 | 0.1552195 | −0.0160021 |
| 88 | cg11207983 | −1.1081970 | 0.0136482 | −0.0151249 |
| 89 | cg23094836 | 0.1839740 | 0.0816395 | 0.0150195 |
| 90 | cg18168310 | 0.1812477 | 0.0787177 | 0.0142674 |
| 91 | cg27607283 | −0.1869241 | 0.0675710 | −0.0126306 |
| 92 | cg15224432 | 0.1000018 | 0.1255512 | 0.0125553 |
| 93 | cg23983141 | 0.3113598 | 0.0382628 | 0.0119135 |
| 94 | cg03412308 | 0.3670085 | 0.0283964 | 0.0104217 |
| 95 | cg26376705 | −0.1683319 | 0.0610850 | −0.0102826 |
| 96 | cg22610434 | 0.0624740 | 0.1636201 | 0.0102220 |
| 97 | cg07360731 | 0.3232514 | 0.0295691 | 0.0095582 |
| 98 | cg00237268 | 0.0967767 | 0.0881654 | 0.0085324 |
| 99 | cg26091142 | 0.1823433 | 0.0461166 | 0.0084091 |
| 100 | cg08871354 | −0.3763483 | 0.0200765 | −0.0075558 |
| 101 | cg10369169 | 0.2043032 | 0.0367151 | 0.0075010 |
| 102 | cg20016411 | 0.0977677 | 0.0725029 | 0.0070884 |
| 103 | cg22247041 | 0.0896301 | 0.0684140 | 0.0061320 |
| 104 | cg26330577 | −0.0645760 | 0.0821321 | −0.0053038 |
| 105 | cg21309167 | −0.0211921 | 0.2460257 | −0.0052138 |
| 106 | cg20103938 | −0.1465496 | 0.0326266 | −0.0047814 |
| 107 | cg01207481 | −0.1241352 | 0.0360947 | −0.0044806 |
| 108 | cg00702719 | 0.0675834 | 0.0645173 | 0.0043603 |
| 109 | cg17362321 | −0.5611777 | 0.0076768 | −0.0043080 |
| 110 | cg07545140 | −0.1117111 | 0.0272461 | −0.0030437 |
| 111 | cg23724447 | 0.1420993 | 0.0213484 | 0.0030336 |
| 112 | cg10144493 | −0.0392983 | 0.0699794 | −0.0027501 |
| 113 | cg08105590 | −0.0468810 | 0.0579803 | −0.0027182 |
| 114 | cg07626033 | 0.0275300 | 0.0957723 | 0.0026366 |
| 115 | cg11438552 | 0.0230252 | 0.0833171 | 0.0019184 |
| 116 | cg14911395 | −0.0233554 | 0.0613505 | −0.0014329 |
| 117 | cg15507719 | 0.0216813 | 0.0634193 | 0.0013750 |
| 118 | cg12409525 | −0.0175702 | 0.0637998 | −0.0011210 |
| 119 | cg00151370 | 0.0042888 | 0.0721145 | 0.0003093 |
| 120 | cg18299068 | 0.0038156 | 0.0600407 | 0.0002291 |
| 121 | cg03846111 | −0.0029002 | 0.0686970 | −0.0001992 |
| | (Intercept) | −16.2893801 | NA | NA |

TABLE 5
list of 24 CpG sites (CpG sites from “serology used”
(list of 16) model and “serology not used” (list of
20) model combined), ranked by standardized coefficient, and intercept

| CpG site | | | | Standardized |
|---|---|---|---|---|
| number | feature | coefficient | SD | Coefficients |
| 1 | cg20843080 | 15.77687026 | 0.029859042 | 0.471082235 |
| 2 | cg20000994 | 84.90400942 | 0.004587033 | 0.389457483 |
| 3 | cg09591303 | 28.97596697 | 0.012935949 | 0.374831639 |
| 4 | cg26329816 | 35.30055013 | 0.010337022 | 0.364902577 |
| 5 | cg03332314 | 106.9202383 | 0.003334964 | 0.356575105 |
| 6 | cg01962018 | 43.27390695 | 0.008179354 | 0.353952607 |
| 7 | cg24009030 | 16.52494427 | 0.021174452 | 0.349906646 |
| 8 | cg12876900 | 16.41978794 | 0.020012705 | 0.328604375 |
| 9 | cg08669718 | 51.46063393 | 0.006261871 | 0.322239864 |
| 10 | cg07777224 | 97.10036013 | 0.003248312 | 0.315412233 |
| 11 | cg09444426 | 19.83301631 | 0.014809101 | 0.293709135 |
| 12 | cg04314318 | 18.3716831 | 0.014747527 | 0.270936901 |
| 13 | cg24789321 | −12.05051034 | 0.020858152 | −0.251351372 |
| 14 | cg03561638 | 3.400185379 | 0.072827414 | 0.247626708 |
| 15 | cg18782736 | 167.6401371 | 0.001198402 | 0.200900273 |
| 16 | cg04373285 | 6.845601821 | 0.029103723 | 0.199232502 |
| 17 | cg14369970 | 163.5769483 | 0.001207539 | 0.197525562 |
| 18 | cg05300717 | −5.746549576 | 0.034350669 | −0.197397821 |
| 19 | cg04399899 | 2.364502616 | 0.081552985 | 0.192832247 |
| 20 | cg07930752 | −3.708677037 | 0.049239709 | −0.182614179 |
| 21 | cg27552857 | 2.28933518 | 0.050053837 | 0.114590009 |
| 22 | cg07329251 | 1.818008793 | 0.061296839 | 0.111438192 |
| 23 | cg10266904 | −1.92218009 | 0.042281234 | −0.081272146 |
| 24 | cg21845373 | 2.850846199 | 0.028476201 | 0.081181268 |
| | (Intercept) | −100.9269723 | #N/A | #N/A |

TABLE 6
list of 20 CpG sites of “serology not used” model,
ranked by standardised coefficient, and intercept

| CpG site | | | | Standardized |
|---|---|---|---|---|
| number | feature | coefficient | SD | Coefficients |
| 1 | cg20843080 | 13.26699 | 0.029859 | 0.396139536 |
| 2 | cg12876900 | 8.970328 | 0.020013 | 0.179520531 |
| 3 | cg07777224 | 42.69172 | 0.003248 | 0.138676007 |
| 4 | cg27552857 | 1.755222 | 0.050054 | 0.087855573 |
| 5 | cg08669718 | 12.80639 | 0.006262 | 0.08019197 |
| 6 | cg10266904 | −1.60389 | 0.042281 | −0.067814535 |
| 7 | cg20000994 | 13.98844 | 0.004587 | 0.06416545 |
| 8 | cg24009030 | 2.861653 | 0.021174 | 0.06059393 |
| 9 | cg14369970 | 46.09088 | 0.001208 | 0.055656541 |
| 10 | cg07930752 | −0.92511 | 0.04924 | −0.045552038 |
| 11 | cg04399899 | 0.552604 | 0.081553 | 0.045066469 |
| 12 | cg05300717 | −1.2297 | 0.034351 | −0.042240992 |
| 13 | cg09591303 | 2.998086 | 0.012936 | 0.038783092 |
| 14 | cg21845373 | 1.031514 | 0.028476 | 0.02937361 |
| 15 | cg26329816 | 1.799846 | 0.010337 | 0.018605048 |
| 16 | cg03332314 | 5.330675 | 0.003335 | 0.017777607 |
| 17 | cg09444426 | 1.009077 | 0.014809 | 0.014943517 |
| 18 | cg24789321 | −0.52829 | 0.020858 | −0.011019068 |
| 19 | cg03561638 | 0.004446 | 0.072827 | 0.00032376 |
| 20 | cg01962018 | 0.008244 | 0.008179 | 6.74315E−05 |
| | (Intercept) | −19.1612 | #N/A | #N/A |

TABLE 7
list of 16 CpG sites of “serology used” model,
ranked by standardised coefficient together with serology data
(“ccp_pos” receives a value of 1 if anti-CCP antibodies
are present, or 0 if not present; the same applies for “
RF_pos” in respect of rheumatoid factor), and intercept

| CpG site | | | | Standardized |
|---|---|---|---|---|
| number | feature | coefficient | SD | Coefficients |
| | ccp_pos | 1.260216476 | 0.446932212 | 0.563231337 |
| 1 | cg20843080 | 8.666117466 | 0.029859042 | 0.258761967 |
| 2 | cg12876900 | 4.80227534 | 0.020012705 | 0.09610652 |
| 3 | cg20000994 | 20.80938106 | 0.004587033 | 0.095453315 |
| 4 | cg05300717 | −2.685841461 | 0.034350669 | −0.092260451 |
| 5 | cg07777224 | 22.29207398 | 0.003248312 | 0.072411604 |
| 6 | cg14369970 | 53.22376723 | 0.001207539 | 0.06426978 |
| 7 | cg27552857 | 1.268899475 | 0.050053837 | 0.063513287 |
| 8 | cg21845373 | 2.210246582 | 0.028476201 | 0.062939425 |
| 9 | cg08669718 | 8.708561293 | 0.006261871 | 0.054531889 |
| 10 | cg10266904 | −1.182741468 | 0.042281234 | −0.050007769 |
| 11 | cg07329251 | 0.515092387 | 0.061296839 | 0.031573535 |
| | RF_pos | 0.073348065 | 0.416084865 | 0.03051902 |
| 12 | cg04314318 | 1.795493458 | 0.014747527 | 0.026479089 |
| 13 | cg04399899 | 0.224203509 | 0.081552985 | 0.018284465 |
| 14 | cg07930752 | −0.263108579 | 0.049239709 | −0.01295539 |
| 15 | cg04373285 | 0.126532609 | 0.029103723 | 0.00368257 |
| 16 | cg18782736 | 0.58870733 | 0.001198402 | 0.000705508 |
| | (Intercept) | −10.15560912 | #N/A | #N/A |

TABLE 9
list of 145 CpG sites identified as relevant for the identification of rheumatoid arthritis
(lists of 121 and 24 combined), including associated biological information

| CpG site | | | | | |
|---|---|---|---|---|---|
| number | feature | chr | UCSC_RefGene_Name | pos | Relation_to_Island |
| 1 | cg16411857 | chr16 | NLRC5 | 57023191 | Island |
| 2 | cg22898082 | chr19 | SMARCA4 | 11074428 | S_Shelf |
| 3 | cg05428452 | chr6 | HLA-DQA2 | 32712979 | OpenSea |
| 4 | cg04708340 | chr19 | SAFB, SAFB2 | 5622366 | Island |
| 5 | cg13714271 | chr9 | SMU1 | 33076774 | Island |
| 6 | cg14950044 | chr20 | BCAS4 | 49457327 | Island |
| 7 | cg19878200 | chr11 | TH | 2187421 | N_Shore |
| 8 | cg10003549 | chr20 | KIF16B | 16554254 | Island |
| 9 | cg00966255 | chr16 | | 10479562 | N_Shore |
| 10 | cg11164639 | chr15 | | 91192950 | OpenSea |
| 11 | cg26164488 | chr2 | | 64440295 | OpenSea |
| 12 | cg24514600 | chr8 | PVT1 | 128805414 | N_Shore |
| 13 | cg27008565 | chr8 | NCALD | 103135208 | N_Shore |
| 14 | cg27095222 | chr11 | | 88090861 | OpenSea |
| 15 | cg02030958 | chr13 | | 110386267 | OpenSea |
| 16 | cg13651908 | chr2 | CD28 | 204570033 | OpenSea |
| 17 | cg21486694 | chr19 | ALDH16A1 | 49957559 | S_Shore |
| 18 | cg09873215 | chr10 | CNNM2 | 104687348 | OpenSea |
| 19 | cg25560009 | chr8 | | 144303218 | OpenSea |
| 20 | cg04917446 | chr17 | HOXB9 | 46699073 | Island |
| 21 | cg21889472 | chr5 | | 42992555 | Island |
| 22 | cg13081526 | chr6 | | 32449961 | OpenSea |
| 23 | cg19945060 | chr8 | | 41508779 | S_Shore |
| 24 | cg08082299 | chr16 | E4F1 | 2284125 | Island |
| 25 | cg14026109 | chr6 | MICAL1 | 109775014 | N_Shore |
| 26 | cg06387622 | chr6 | LOC285768 | 1100823 | OpenSea |
| 27 | cg00119117 | chr20 | INSM1 | 20349621 | Island |
| 28 | cg03441250 | chr17 | | 2649784 | N_Shelf |
| 29 | cg19544767 | chr13 | | 50414468 | OpenSea |
| 30 | cg09455126 | chr15 | SNORD116-24 | 25338790 | OpenSea |
| 31 | cg07231053 | chr1 | GALNT2 | 230399402 | OpenSea |
| 32 | cg01534423 | chr17 | | 18965556 | Island |
| 33 | cg23985214 | chr17 | SOCS3 | 76356261 | Island |
| 34 | cg08447792 | chr4 | CCDC109B | 110600020 | OpenSea |
| 35 | cg11646192 | chr4 | EREG | 75230615 | OpenSea |
| 36 | cg14052235 | chr19 | ELANE | 853145 | Island |
| 37 | cg18036763 | chr22 | PHF21B | 45404910 | Island |
| 38 | cg26043955 | chr8 | | 123095289 | OpenSea |
| 39 | cg20726993 | chr3 | LOC151658 | 107565406 | OpenSea |
| 40 | cg06544989 | chr22 | UNC84B | 39130855 | OpenSea |
| 41 | cg12359114 | chr11 | NAA40 | 63706246 | Island |
| 42 | cg05370853 | chr6 | HLA-DQA1 | 32606634 | OpenSea |
| 43 | cg12192813 | chr6 | HLA-DQB1 | 32632108 | N_Shore |
| 44 | cg22424532 | chr7 | ASZ1 | 117067727 | S_Shore |
| 45 | cg02539793 | chr6 | | 87861586 | Island |
| 46 | cg00672574 | chr17 | MRPL12 | 79669841 | Island |
| 47 | cg18734095 | chr19 | NOSIP | 50062005 | S_Shore |
| 48 | cg01120173 | chr17 | ZNF232 | 5019669 | Island |
| 49 | cg14480789 | chr3 | NKTR | 42642589 | Island |
| 50 | cg21106695 | chr14 | | 97370064 | OpenSea |
| 51 | cg01849466 | chr14 | ZFYVE21 | 104193079 | N_Shore |
| 52 | cg24931954 | chr11 | OSBPL5 | 3164851 | OpenSea |
| 53 | cg15674266 | chr17 | STX8 | 9153825 | OpenSea |
| 54 | cg05225373 | chr14 | DLGAP5 | 55657967 | N_Shore |
| 55 | cg16078521 | chr1 | ZSWIM5 | 45672383 | Island |
| 56 | cg15996459 | chr17 | RPH3AL | 184106 | N_Shore |
| 57 | cg25666547 | chr8 | | 144432352 | OpenSea |
| 58 | cg20000464 | chr11 | C11orf30 | 76261997 | OpenSea |
| 59 | cg06118351 | chr16 | C16orf71 | 4788808 | N_Shore |
| 60 | cg00249093 | chr19 | | 5804642 | Island |
| 61 | cg22125253 | chr11 | OR10G4 | 123886957 | OpenSea |
| 62 | cg14883135 | chr2 | | 43051175 | N_Shelf |
| 63 | cg19057882 | chr20 | RALGAPB | 37101373 | Island |
| 64 | cg17131070 | chr1 | SDCCAG8, CEP170 | 243418713 | Island |
| 65 | cg19273683 | chr1 | ECE1 | 21656047 | OpenSea |
| 66 | cg08707123 | chr16 | SOLH | 589874 | N_Shore |
| 67 | cg19204693 | chr11 | LRP5 | 68206027 | Island |
| 68 | cg13862851 | chr2 | NGEF | 233791890 | Island |
| 69 | cg11628021 | chr19 | | 17491382 | S_Shelf |
| 70 | cg11925488 | chr11 | SLC35C1 | 45825579 | N_Shore |
| 71 | cg05522848 | chr5 | RAD17, MARVELD2 | 68710359 | N_Shore |
| 72 | cg24702253 | chr11 | MRGPRG, C11orf36 | 3240068 | S_Shore |
| 73 | cg17903019 | chr7 | FBXL18 | 5552472 | N_Shore |
| 74 | cg21853806 | chr17 | SLC16A5 | 73096475 | OpenSea |
| 75 | cg21247338 | chr2 | | 129548448 | OpenSea |
| 76 | cg05494709 | chr12 | LOC653113 | 8396767 | S_Shore |
| 77 | cg26720338 | chr16 | JPH3 | 87635575 | N_Shore |
| 78 | cg09138892 | chr17 | FLJ90757 | 79005662 | N_Shore |
| 79 | cg14882966 | chr2 | | 3699353 | Island |
| 80 | cg26561212 | chr7 | | 157255200 | OpenSea |
| 81 | cg26019250 | chr11 | | 44335005 | N_Shelf |
| 82 | cg06311103 | chr13 | | 31579287 | OpenSea |
| 83 | cg01437439 | chr1 | SPSB1 | 9383245 | OpenSea |
| 84 | cg10905401 | chr10 | GPR120 | 95327292 | S_Shore |
| 85 | cg05322982 | chr17 | NMT1 | 43186129 | OpenSea |
| 86 | cg01558909 | chr16 | HBM | 215845 | Island |
| 87 | cg23574427 | chr4 | BBS7, CCNA2 | 122746245 | S_Shore |
| 88 | cg11207983 | chr1 | C1orf51 | 150254477 | Island |
| 89 | cg23094836 | chr7 | | 155240306 | N_Shore |
| 90 | cg18168310 | chr11 | | 68920771 | N_Shelf |
| 91 | cg27607283 | chr5 | | 15385796 | OpenSea |
| 92 | cg15224432 | chr19 | IGFL3 | 46628367 | OpenSea |
| 93 | cg23983141 | chr8 | TRAPPC9 | 140989917 | OpenSea |
| 94 | cg03412308 | chr5 | MAST4 | 66459594 | Island |
| 95 | cg26376705 | chrX | SPANXC | 140336617 | OpenSea |
| 96 | cg22610434 | chr1 | CD1C | 158259914 | OpenSea |
| 97 | cg07360731 | chr12 | MMP17 | 132334499 | Island |
| 98 | cg00237268 | chr4 | SORCS2 | 7287615 | OpenSea |
| 99 | cg26091142 | chr15 | ISLR2, LOC283731 | 74423077 | S_Shore |
| 100 | cg08871354 | chr16 | GINS2 | 85723489 | S_Shore |
| 101 | cg10369169 | chr17 | NLGN2 | 7312041 | Island |
| 102 | cg20016411 | chr11 | DRD2 | 113344987 | N_Shore |
| 103 | cg22247041 | chr17 | | 73004677 | N_Shelf |
| 104 | cg26330577 | chr6 | MICB | 31474279 | OpenSea |
| 105 | cg21309167 | chr4 | PDGFRA | 55143285 | OpenSea |
| 106 | cg20103938 | chr4 | ELF2 | 140005223 | Island |
| 107 | cg01207481 | chrX | WBP5 | 102611843 | OpenSea |
| 108 | cg00702719 | chr6 | | 74025019 | Island |
| 109 | cg17362321 | chr22 | DGCR5 | 18958316 | Island |
| 110 | cg07545140 | chr2 | TTC15 | 3468531 | OpenSea |
| 111 | cg23724447 | chr10 | BUB3 | 124913897 | Island |
| 112 | cg10144493 | chr6 | SYNGAP1 | 33395133 | N_Shore |
| 113 | cg08105590 | chr16 | FAM38A | 88849599 | N Shore |
| 114 | cg07626033 | chr3 | SLC6A1 | 11036898 | S_Shore |
| 115 | cg11438552 | chr22 | PRODH | 18919803 | N_Shelf |
| 116 | cg14911395 | chr3 | SEMA3B | 50311213 | Island |
| 117 | cg15507719 | chr18 | BRUNOL4 | 34837160 | S_Shelf |
| 118 | cg12409525 | chr21 | DSCAM | 42219211 | Island |
| 119 | cg00151370 | chr6 | ATXN1 | 16323285 | N_Shelf |
| 120 | cg18299068 | chr4 | MAEA | 1305425 | S_Shore |
| 121 | cg03846111 | chr12 | ATF7IP | 14547229 | OpenSea |
| 122 | cg20843080 | chr12 | DYNLL1 | 120925596 | OpenSea |
| 123 | cg12876900 | chr11 | IFITM3 | 320732 | S_Shore |
| 124 | cg07777224 | chr19 | ZNF584 | 58919807 | Island |
| 125 | cg20000994 | chr6 | ZBTB12 | 31869553 | Island |
| 126 | cg05300717 | chr11 | DKFZp761E198 | 65546210 | N_Shore |
| 127 | cg27552857 | chr19 | SEMA6B | 4542866 | N_Shore |
| 128 | cg08669718 | chr4 | ZNF718 | 121568 | N_Shelf |
| 129 | cg10266904 | chr16 | NOMO2 | 18570097 | N_Shelf |
| 130 | cg14369970 | chr1 | PGBD2 | 249200561 | Island |
| 131 | cg21845373 | chr21 | BACE2 | 42557262 | OpenSea |
| 132 | cg24009030 | chr13 | | 114912327 | N_Shore |
| 133 | cg07930752 | chr2 | CD28 | 204569955 | OpenSea |
| 134 | cg04399899 | chr4 | RAPGEF2 | 160189254 | OpenSea |
| 135 | cg09591303 | chr13 | TFDP1 | 114243660 | OpenSea |
| 136 | cg07329251 | chr11 | AMPD3 | 10476662 | S_Shelf |
| 137 | cg04314318 | chr1 | SH3BP5L | 249112632 | OpenSea |
| 138 | cg26329816 | chr1 | | 147994129 | Island |
| 139 | cg03332314 | chr8 | C8orf33 | 146278003 | Island |
| 140 | cg09444426 | chr5 | PLEKHG4B | 160518 | OpenSea |
| 141 | cg24789321 | chr1 | ASH1L | 155428192 | OpenSea |
| 142 | cg04373285 | chr16 | UBE2MP1 | 34404619 | Island |
| 143 | cg18782736 | chr22 | | 50982818 | Island |
| 144 | cg03561638 | chr1 | | 198741166 | OpenSea |
| 145 | cg01962018 | chr3 | SLC25A38 | 39424819 | Island |

1. A method of screening for rheumatoid arthritis in a subject, the method comprising using the methylation levels of at least:
4, 5, 10, 20 or 23 CpG sites selected from the list of CpG site numbers 1 to 145 of Table 9;
in DNA from a biological sample obtained from the subject in order to screen for rheumatoid arthritis in the subject, wherein said methylation levels are used to provide an indication of the presence or absence of rheumatoid arthritis in the subject.

2. The method of claim 1, wherein the at least 4, 5, 10, 20 or 23 CpG sites selected from the list of CpG site numbers 1 to 145 of Table 9 comprise at least:
5, 10, 15, 20 or 24 CpG sites selected from the list of CpG site numbers 1 to 24 of Table 5; and/or
4, 5, 10, 11, 20, 30 or 31 CpG sites selected from the list of CpG site numbers 1 to 121 of Table 3.

3. The method of claim 2, wherein the at least 4, 5, 10, 11, 20, 30 or 31 CpG sites selected from the list of CpG site numbers 1 to 121 of Table 3 comprise at least 5, 10, 20, 30 or 31 CpG sites selected from the list of CpG site numbers 1 to 31 of Table 3; and/or
wherein the at least 5, 10, 15, 20 or 24 CpG sites selected from the list of CpG site numbers 1 to 24 of Table 5 comprise at least:
5, 10, 15, or 20 CpG sites selected from the list of CpG site numbers 1 to 20 of Table 6; and/or
5, 10, 15, or 16 CpG sites selected from the list of CpG sites numbers 1 to 16 of Table 7.

4. (canceled)

5. The method of claim 1, comprising using the rheumatoid factor status and/or the anti-citrullinated protein antibody status of the subject in addition to said methylation levels in order to provide said indication.

6. The method of claim 1, comprising calculating a likelihood of the subject having rheumatoid arthritis as a function of said methylation levels.

7. The method of claim 6, comprising calculating the likelihood as a function of a linear combination of said methylation levels, preferably wherein the linear combination of said methylation levels comprises a weighted sum of said methylation levels; and/or
comprising calculating the likelihood as a logistic function of a linear combination of said methylation levels; and/or
comprising receiving data representative of said methylation levels, and inputting the data to an algorithm for evaluating said function to determine the likelihood of the subject having rheumatoid arthritis.

8-9. (canceled)

10. The method of claim 1, further comprising making a diagnosis of rheumatoid arthritis based on the methylation levels, and/or based on a likelihood of the subject having rheumatoid arthritis that has been calculated as a function of the methylation levels, optionally by comparing the methylation levels or likelihood with a cutoff value.

11. The method of claim 1, wherein said subject is a subject at risk of developing rheumatoid arthritis, or is a subject having or suspected of having rheumatoid arthritis.

12. The method of claim 1, wherein
the biological sample is a blood sample, or a white blood cell sample.

13. The method of claim 1, further comprising reporting the results of the method, optionally by preparing a written or electronic report.

14. The method of claim 1, wherein the method is implemented by a computer.

15. The method of claim 1, wherein said method comprises a step of measuring the methylation levels before the step of using the methylation levels.

16. A computer program comprising instructions that, when executed by a processing system, cause the processing system to process data representative of methylation levels of at least:
4, 5, 10, 20 or 23 CpG sites selected from the list of CpG site numbers 1 to 145 of Table 9;
in DNA from a biological sample obtained from a subject, to calculate a likelihood of the subject having rheumatoid arthritis.

17. The computer program of claim 16, wherein the at least 4, 5, 10, 20 or 23 CpG sites selected from the list of CpG site numbers 1 to 145 of Table 9 comprise at least:
4, 5, 10, 11, 20, 30 or 31 CpG sites selected from the list of CpG site numbers 1 to 121 of Table 3; and/or
5, 10, 15, 20 or 24 CpG sites selected from the list of CpG site numbers 1 to 24 of Table 5.

18. The computer program of claim 17, wherein the at least 4, 5, 10, 11, 20, 30 or 31 CpG sites selected from the list of CpG site numbers 1 to 121 of Table 3 comprise at least 5, 10, 20, 30 or 31 CpG sites selected from the list of CpG site numbers 1 to 31 of Table 3; and/or
wherein the at least 5, 10, 15, 20 or 24 CpG sites selected from the list of CpG site numbers 1 to 24 of Table 5 comprise at least:
5, 10, 15, or 20 CpG sites selected from the list of CpG site numbers 1 to 20 of Table 6; and/or
5, 10, 15, or 16 CpG sites selected from the list of CpG sites numbers 1 to 16 of Table 7.

19. (canceled)

20. The computer program of claim 16, comprising instructions that, when executed by a processing system, cause the processing system to process data representative of the rheumatoid factor status and/or the anti-citrullinated protein antibody status of the subject, in addition to the data representative of the methylation levels.

21. A kit for screening for rheumatoid arthritis in a subject, said kit comprising probes for detecting the methylation levels of at least:
4, 5, 10, 20 or 23 CpG sites selected from the list of CpG site numbers 1 to 145 of Table 9;
in DNA from a biological sample obtained from the subject, wherein the CpG probe component of the kit consists of probes for detecting the methylation levels of up to 500 CpG sites.

22. The kit of claim 21, wherein the at least 4, 5, 10, 20 or 23 CpG sites selected from the list of CpG site numbers 1 to 145 of Table 9 comprise at least:
4, 5, 10, 11, 20, 30 or 31 CpG sites selected from the list of CpG site numbers 1 to 121 of Table 3; and/or
5, 10, 15, 20 or 24 CpG sites selected from the list of CpG site numbers 1 to 24 of Table 5.

23. The kit of claim 22, wherein the at least 4, 5, 10, 11, 20, 30 or 31 CpG sites selected from the list of CpG site numbers 1 to 121 of Table 3 comprise at least 5, 10, 20, 30 or 31 CpG sites selected from the list of CpG site numbers 1 to 31 of Table 3; and/or
wherein the at least 5, 10, 15, 20 or 24 CpG sites selected from the list of CpG site numbers 1 to 24 of Table 5 comprise at least:
5, 10, 15, or 20 CpG sites selected from the list of CpG site numbers 1 to 20 of Table 6; and/or
5, 10, 15, or 16 CpG sites selected from the list of CpG sites numbers 1 to 16 of Table 7.

24. (canceled)

25. The kit of claim 21, comprising a means for detecting rheumatoid factor, and/or a means for detecting anti-citrullinated protein antibody (ACPA).

26. (canceled)
